(19) United States
(12) Patent Application Publication
(10) Pub. No.: US 2024/0238614 A1
(43) Pub. Date: Jul. 18, 2024

(54) LED-Based Phototherapy Systems and Associated Methods

(71) Applicant: Spectrum Medical Technologies, Inc., Vero Beach, FL (US)

(72) Inventors: Mark J. Gerber, Vero Beach, FL (US); Mark Boies, Dagsboro, DE (US)

(73) Assignee: Spectrum Medical Technologies, Inc., Vero Beach, FL (US)

(21) Appl. No.: 18/437,534

(22) Filed: Feb. 9, 2024

Related U.S. Application Data
(63) Continuation-in-part of application No. PCT/US2022/039147, filed on Aug. 2, 2022.
(60) Provisional application No. 63/232,511, filed on Aug. 12, 2021.

(51) Int. Cl. A61N 5/06

(52) U.S. Cl. CPC A61N 5/0624; A61N 2005/0628; A61N 2005/0642; A61N 2005/0644; A61N 2005/0651; A61N 2005/0659; A61N 2005/0661; A61N 2005/0662; A61N 2005/0666; A61N 2005/0667

(57) ABSTRACT

LED-based assemblies/systems and methods for infection prevention and treatment are disclosed. The assemblies/systems include an optical head unit including LEDs and LED drivers, the LED drivers configured to control emission of light from the LEDs at one or more selected wavelengths. The assemblies/systems also include a base that selectively and operatively couples to the optical head unit to provide a consistent distance to a target site and prevent light emitted from the LEDs from escaping into an environment surrounding the target site. A phototherapy method for treating tissue includes (i) connecting an optical head unit to a base, (ii) disposing the base to surround a target site; and (ii) controlling output of LEDs in the optical head unit to emit light through the base at one or more selected wavelengths, the base preventing the light emitted from the LEDs from escaping into an environment surrounding the target site.

### RELATED APPLICATIONS

The present application claims priority to and the benefit of PCT Application No. PCT/US2022/039147, filed on Aug. 2, 2022, which application claimed priority to and the benefit of U.S. Provisional Patent Application No. 63/232,511, filed on Aug. 12, 2021, the disclosures of which are incorporated by reference herein in their entireties.

### FIELD OF THE DISCLOSURE

The present disclosure relates to LED-based phototherapy assemblies/systems and associated methods. More particularly, the present disclosure relates to LED-based phototherapy assemblies/systems and associated methods to prevent and/or treat infections including, inter alia, bacterial infections (including antibiotic-resistant bacterial infections), viral infections and fungal infections.

### BACKGROUND

The lack of new classes of antibiotics in development combined with the declining effectiveness of antibiotics currently in use has prompted the United States government to seek alternatives to antibiotic therapy. Phototherapy in the ultraviolet C (UVC) wavelength spectrum has long been demonstrated as an effective tool for in vitro inactivation of pathogens including bacteria, viruses and fungi. (Dai et al., “Ultraviolet C light for Acinetobacter baumannii wound infections in mice: Potential use for battlefield wound decontamination,” J Trauma Acute Care Surg. (2012 September), 73(3): 661-667 (“Dai I”); Dai et al., “Ultraviolet-C light for treatment of Candida albicans burn infection in mice,” Photochem Photobiol. (2011), 87(2): 342-349, doi: 10.1111/j.1751-1097.2011.00886.x (“Dai II”); Stroud et al., “An investigation into the inhibitory effect of ultraviolet radiation on Trichophyton rubrum,” Lasers in medical science (2013) doi: 29. 10.1007/s10103-013-1287-4; Welch et al., “Far-UVC light: A new tool to control the spread of airborne-mediated microbial diseases,” Sci Rep. (2018), 8(1):2752, (2018) doi:10.1038/s41598-018-21058-w; Narita et al., “Disinfection and healing effects of 222-nm UVC light on methicillin-resistant Staphylococcus aureus infection in mouse wounds” J Photochem Photobiol B. (2018 Apr. 3), doi:10.1016/j.jphotobiol.2017.10.030; Thai et al., “Effect of ultraviolet light C on bacterial colonization in chronic wounds,” Ostomy Wound Manage. (2005); 51(10):32-45; Lytle et al., “Predicted inactivation of viruses of relevance to biodefense by solar radiation,” J Virol. (2005), 79(22): 14244-14252, doi: 10.1128/JVI.79.22.14244-14252.2005; Ernest et al., “SARS-CoV-2 UV Dose-Response Behavior” White Paper Prepared for IUVA; Kim et al., “UVC LED Irradiation Effectively Inactivates Aerosolized Viruses, Bacteria, and Fungi in a Chamber-Type Air Disinfection System,” Appl Environ Microbiol. (2018), 84(17):e00944-18, doi:10.1128/AEM.00944-18). Specifically, the range of light wavelengths from 207-254 nanometers (nm) has been shown to be effective in vitro against known pathogens, including antibiotic resistant bacteria, viruses and fungal infections (Dai I, Dai II, Stroud et al., Welch et al.). The UVC disrupts the deoxyribonucleic acid (DNA) and, once it is altered, pathogens cannot replicate nor can they maintain homeostasis leading to pathogen death. (Buonanno et al., “207-nm UV Light—A Promising Tool for Safe Low-Cost Reduction of Surgical Site Infections,” I: In Vitro Studies PLoS ONE 8(10): e76968. doi: 10. 1371/journal.pone.0076968; Dai I). In addition, UVC energy can interfere with cytoplasmic protein synthesis of pathogens resulting in the inability of the pathogen to maintain homeostasis.

Within the UVC wavelength spectrum, 254 nm and 207/222 nm have been studied at Harvard University (on mouse tissue culture using a mercury vapor lamp at 1 6 milliWatts per square centimeters (mW/cm²)) and Columbia University (excimer lamp 0.33 mW/cm² on human fibroblast tissue culture), respectively. (Dai I; Buonanno et al.). Results of these studies have demonstrated that phototherapy via direct UVC illumination using longer wavelengths at the 254 nm level is greater than 90% effective in deactivation of various pathogens including methicillin resistant staph aureus (MRSA), Actinobacter and Candida. (Dai I; Dai II; Dai et al. “Ultraviolet C irradiation: an alternative antimicrobial approach to localized infections?” Expert Rev Anti Infect Ther. (2012); 10(2): 185-195 (“Dai III”)). However, at the 254 nm wavelength, thymidine dimers were detected in healthy host tissue. These dimers signal potential long term DNA damage to otherwise healthy tissue and effectively prohibits the use of the 254 nm wavelength as a clinical tool.

In contrast, exposing human tissue culture to 207 nm and 222 nm wavelengths did not result in any significant host tissue mutations. However, these wavelengths were significantly less effective in pathogen (including MRSA) neutralization, and biofilm penetration. (Buonanno et al.; Narita et al.).

When it comes to treating wounds, medical practitioners must currently take samples from the wound site and wait 48 to 72 hours for lab results before prescribing antibiotics. This wastes valuable time. The other alternative is for medical practitioners to guess what the offending pathogen is and take a shotgun approach, administering a myriad of antibiotics to attack the suspected pathogen. This approach may not only delay appropriate treatment but can compound antibiotic resistance as the pathogens are exposed to different antibiotics developing a resistance to them. Accordingly, phototherapy systems can be used to avoid these pitfalls and provide immediate and effective treatment of wounds, particularly chronic non-healing wounds, by decontaminating the target area while leaving host tissue without significant mutations.

However, small lamps and other light-emitting structures can be unnecessarily costly, complicated, and fragile. These assemblies typically require bulbs or fiber-optic elements, as well as a source of power which potentially becomes an electric shock risk to the patient. Not only do these assemblies have multiple connections and bulky fixtures that complicate surgery, they can be problematic sources of heat. Also, a light fixture that incorporates elements, such as light bulb(s), into a complex structure generally entails significant manufacturing costs. Further, these light-emitting structures may be difficult to transport and not rated for field use where they may be needed most, e.g., mobile army surgical hospitals or home health visits.

Moreover, light sources (whether overhead or localized) can cause glares and reflections that may be blinding, distracting or otherwise impede visual acuity. For example, light that glares off of monitors interferes with the ability of medical staff to monitor a patient's vital signs, and light reflecting from steel or mirrored surfaces and instruments can interfere with a surgeon's focus.

Furthermore, LEDs operating at certain wavelengths, in particular UVC, are inefficient whereby most of the energy, e.g., electricity, being transferred to the LEDs is converted into heat rather than photons, i.e., light. This can present thermodynamic challenges, since as discussed herein, it is desirable to prevent overheating of the circuitry and system (e.g., maintaining an external operating temperature of the system below 40° C.). The LEDs may not only burn out at threshold temperatures but there is also an inverse correlation between heat generation and photon output from the LEDs, i.e., at higher temperatures the LEDs produce less light. Accordingly, heat management of such phototherapy systems is critically important.

Thus, there exists a need for assemblies/systems and associated methods that are clinically effective in preventing and/or treating infections including, inter alia, bacterial infections (including antibiotic-resistant bacterial infections), viral infections and fungal infections. Moreover, there exists a need for assemblies/systems and associated methods that are clinically effective in preventing and/or treating infections that do not cause potential long term DNA damage to otherwise healthy tissue. Furthermore, there exists a need for assemblies/systems which are designed to be portable and durable so they can be used in operating rooms, mobile army surgical hospitals, or home visits. These and other needs and opportunities are addressed and/or overcome by the assemblies, systems and methods of the present disclosure.

### SUMMARY

The present disclosure provides LED-based assemblies/systems and associated methods that function as an advantageous antisepsis phototherapy platform to address the need for infection prevention and treatment, including prevention/treatment of antibiotic resistant infections. In exemplary embodiments of the disclosed LED-based assemblies/systems and associated methods, surgical site infections (SSI) are effectively addressed through an integrated illuminating and phototherapy system. For the first time a surgeon has the option to decontaminate a wound at will at any time before, during, and/or after a surgical procedure (as opposed to only during pre-surgical preparation of the skin). This is especially important as contamination of an open surgical wound has been shown to be an ongoing process throughout the surgical procedure. (Welch et al.; Orthopaedics & Traumatology: Surgery & Research 101, “Preventing surgical-site infections: Measures other than antibiotics” (2015) S77-S83Available online atScienceDirect www.sciencedirect.comReview).

More specifically, the present disclosure provides a clinical phototherapy platform that significantly neutralizes pathogens by greater than 90% using a relatively short exposure times while steadfastly preserving the health of surrounding host tissue. In addition, the disclosed phototherapy platform may be integrated seamlessly into existing clinical workflows and/or surgical procedures and protocols. Furthermore, it is noted that the application of UV light as disclosed herein can be helpful for medical and non-medical applications (e.g., where there is a risk of re-introduction of microbes and other kinds of contaminants).

Embodiments of phototherapy assemblies/systems and associated methods can additionally provide enhanced visualization of a surgical field by incorporating LEDs which emit light in the visible spectrum in such a manner as to illuminate a target without causing glare in the field or in the operator's eyes.

In accordance with embodiments of the present disclosure, a phototherapy system and associated methods are disclosed. The phototherapy systems and associated methods emit light at wavelengths found throughout the electromagnetic light spectrum, which includes infrared, visible, and ultraviolet light/radiation. As an example, the phototherapy systems and associated methods can emit energy at wavelengths in a range of approximately 100 nm to approximately 10,000 nm. An optical head unit can have a housing that incorporates circuitry, which includes light emitting diodes (LEDs) and LED drivers. The LED drivers can control the LEDs to emit light at one or more wavelengths of the electromagnetic spectrum to illuminate and/or decontaminate, e.g., a surgical site. As an example, the LEDs can be controlled by the LED drivers to emit light in the ultraviolet (UV) or UVC spectrum to provide antisepsis phototherapy for infection prevention and treatment. A base can selectively and operatively couple to the optical head unit to prevent the light emitted from the plurality of LEDs from escaping into an environment surrounding a target site. The LEDs can cease to emit light in response to the optical head unit being detached from the base and/or can be controlled to emit the light for a specified period of time. The LEDs can be arranged to output a total energy of, for example, approximately 100 micro-Joules per square centimeter, although embodiments of the present disclosure can have an energy output that is greater or less than 100 micro-Joules per square centimeter. The LEDs can be arranged in a lattice or other structure (e.g., concentric circles, squares, linear rows and columns, etc.), and/or can form a circular perimeter, rectangular perimeter, triangular perimeter, trapezoidal perimeter, hexagonal perimeter, or a differently shaped perimeter. The LEDs can be calibrated to emit the light with homogeneous light distribution with a spatial uniform density +/−10%. Different sets of the plurality of LEDs are controlled by different ones of the plurality of LED drivers. To maintain a homogeneous pattern of light distribution at a target site, the output of different LEDs in the optical head unit may not be homogeneous or uniform. As an example, the output of the LEDs at the perimeter of the optical head unit can be higher than those in the center of the optical head unit to compensate for a lower density of LEDs at the periphery.

The system can include at least one of a collimator or a filter disposed over the LEDs.

The base can include a shutter that controls an aperture through which the light emitted by the plurality of LEDs passes, wherein the shutter controls at least one of a size of the aperture or a geometry of the aperture.

The circuitry can include a non-transitory computer-readable medium storing instructions and a processor that executes the instructions to output drive signals to the LED drivers to control an operation of the plurality of LEDs. The circuitry can include one or more sensors to sense one or more parameters associated with an operation of the circuitry. The circuitry can cease operation in response to the one or more sensors sensing that one of more of the parameters exceeds a specified threshold. The one or more sensors can include a temperature sensor to sense heat dissipated by the circuitry, a light sensor to sense the light emitted by the plurality of LEDs, and/or a contact sensor between the optical head unit and the base. A temperature at the bottom of the base can remain at less than 40° Celsius during operation of the circuitry.

The base can have an annular configuration. As an example, the base can have an asymmetrical C-shaped cross-sectional profile having an upper portion and a lower portion with the upper portion extending over the lower portion and extending further inwards than the lower portion to define the asymmetrical C-shaped cross-sectional profile. The base can include a light source to deliver light into the base. As another example, the base can have a general shape of a truncated cone that has an inner diameter that generally decrease along the height from the bottom to the top of the base. The base can at least partially surround a pre-determined location and diffuse the light inwards and downwards toward the pre-determined location.

Embodiments of the optical head unit can include a heat sink that transfers heat away from the circuitry, where a portion of the heat sink can form an exterior surface of a housing of the optical head unit. For example, the housing can include a housing bottom and a housing top, where the heat sink is disposed between the housing top and housing bottom. A thermally conductive auxiliary member can be configured to be removable or fixedly attached to the at least a portion of the heat sink forming the exterior surface of the housing. One or more bolts can be used to secure the heat sink to the circuitry to improve heat transfer, the placement/position of the bolts can be determined using a heat map of the circuitry.

Embodiments of the present disclosure can also include a docking station configured to receive the optical head unit, where the docking station can be configured to store the optical head unit between uses, charge a power supply of the optical head unit, and/or transfer heat away from the optical head unit. The optical head unit and the docking station can be configured to charge the power supply of the circuitry in the optical head unit using wireless power transfer. The docking station can include a docking area that receives and retains the optical head unit and a thermally conduct member that is biased by a spring to be in physical contact with the at least a portion of the heat sink forming the exterior surface of the housing when the optical head unit is retained in the docking area. The docking station can also include a fan disposed proximate to the thermally conductive member to cool the thermally conductive member.

Embodiments of the present disclosure can also include a resealable housing of the optical head unit, where the housing can be sealed and unsealed to prevent contamination of the circuitry. There can also be a second optical head unit with a second housing, where the circuitry can be removed from the housing in the optical head unit and incorporated into the second housing of the second optical head unit. The second optical head unit can be selectively and operatively coupled to the base.

In accordance with embodiments of the present disclosure, a phototherapy method for treating infections is disclosed. The method can include connecting an optical head unit to a base; controlling an output of a LEDs to emit light at a wavelength in the ultraviolet spectrum to provide antisepsis phototherapy for infection prevention and treatment. The base can prevent the light emitted from the LEDs from escaping into an environment surrounding a target site. The LEDs emit the light with homogeneous light distribution with a spatial uniform density within ten percent (+/−10%) and/or (ii) output a total energy of approximately 100 micro-Joules per square centimeter, although embodiments of the present disclosure can have an energy output (irradiance) that is greater or less than 100 micro-Joules per square centimeter.

Any combination or permutation of embodiments is envisioned. Additional advantageous features, functions and applications of the disclosed systems, methods and assemblies of the present disclosure will be apparent from the description which follows, particularly when read in conjunction with the appended figures. All references listed in this disclosure are hereby incorporated by reference in their entireties.

### BRIEF DESCRIPTION OF THE FIGURES

Features and aspects of embodiments are described below with reference to the accompanying drawings, in which elements are not necessarily depicted to scale.

Exemplary embodiments of the present disclosure are further described with reference to the appended figures. It is to be noted that the various steps, features and combinations of steps/features described below and illustrated in the figures can be arranged and organized differently to result in embodiments which are still within the scope of the present disclosure. To assist those of ordinary skill in the art in making and using the disclosed assemblies, systems and methods, reference is made to the appended figures, wherein:

FIG. 1 depicts an example LED-based phototherapy circuit 100 in accordance with embodiments of the present disclosure;

FIGS. 2A-B depict an example phototherapy system in accordance with embodiments of the present disclosure;

FIG. 3 is a graph illustrating an energy distribution profile of an example LED emitting light at a wavelength of 235 nanometers in accordance with embodiments of the present disclosure;

FIGS. 4A-C depict example LED arrangements of an optical head unit in accordance with embodiments of the present disclosure;

FIGS. 5A-C depict example light distributions for the example LED arrangements of FIGS. 3A-C in accordance with embodiments of the present disclosure;

FIGS. 6A-6C show an exemplary base assembly of the phototherapy system in accordance with embodiments of the present disclosure;

FIG. 7 illustrates use of an exemplary base assembly of the phototherapy system in accordance with embodiments of the present disclosure;

FIG. 8 shows another exemplary phototherapy assembly of the present disclosure;

FIG. 9 is a photograph of an example experimental embodiment of a LED-based phototherapy system in accordance with embodiments of the present disclosure;

FIG. 10 is another photograph of the example experimental embodiment of a LED-based phototherapy system of FIG. 9 in accordance with embodiments of the present disclosure;

FIG. 11A shows a perspective view of an exemplary base assembly of the phototherapy system in accordance with embodiments of the present disclosure;

FIG. 11B shows a side profile of the exemplary base assembly of FIG. 11A;

FIG. 11C shows a cross-section of the exemplary base assembly of FIG. 11B along the line A-A;

FIG. 12A is an exploded view an example embodiment of an optical head unit assembly in accordance with embodiments of the present disclosure;

FIG. 12B is a schematic view of the optical head unit assembly of FIG. 12A in an assembled state and attached to a base assembly in accordance with embodiments of the present disclosure;

FIG. 12C is a perspective view of the optical head unit assembly of FIG. 12A in an assembled state and attached to a base assembly in accordance with embodiments of the present disclosure;

FIG. 12D is a top view of the optical head unit assembly of FIG. 12A in an assembled state and attached to a base assembly in accordance with embodiments of the present disclosure;

FIG. 12E is a bottom view of the optical head unit assembly of FIG. 12A in an assembled state and attached to a base assembly in accordance with embodiments of the present disclosure;

FIG. 12F shows a portion of an example LED circuit that can be included in a base assembly in accordance with embodiments of the present disclosure;

FIG. 12G shows an example configuration of a power switch and battery for the LED circuit in accordance with embodiments of the present disclosure;

FIG. 12H is an alternate bottom view of the optical head unit assembly of FIG. 12A in an assembled state and attached to a base assembly with improved thermal management in accordance with embodiments of the present disclosure;

FIGS. 13A-B are perspective views of an example embodiment of an optical head unit assembly attached to a base assembly in accordance with embodiments of the present disclosure;

FIG. 13C illustrates an example embodiment of the docking station configured to the receive embodiments of an optical head unit assembly;

FIG. 14A shows a partially exploded view of a docking station configured to receive embodiments of an optical head unit in accordance with embodiments of the present disclosure;

FIG. 14B is a perspective view of the docking station of FIG. 13A with an optical head unit positioned in the docking station in accordance with embodiments of the present disclosure;

FIG. 14C is a top view of the docking station of FIG. 13A with an optical head unit positioned in the docking station in accordance with embodiments of the present disclosure; and

FIG. 14D is another perspective view of the docking station of FIG. 13A with an optical head unit positioned in the docking station in accordance with embodiments of the present disclosure.

### DETAILED DESCRIPTION

Example embodiments disclosed herein are illustrative of advantageous phototherapy assemblies, and systems of the present disclosure and methods/techniques thereof. It should be understood, however, that the disclosed embodiments are merely exemplary of the present disclosure, which may be embodied in various forms. Therefore, details disclosed herein with reference to exemplary phototherapy systems and associated methods are not to be interpreted as limiting, but merely as the basis for teaching one skilled in the art how to make and use advantageous phototherapy systems of the present disclosure.

The present disclosure provides improved phototherapy assemblies/systems, and associated methods for using the same. More particularly, the present disclosure provides advantageous phototherapy assemblies/systems that provide light to and/or illuminate/treat predetermined locations (e.g., surgical sites, wound sites, infected areas of a body, areas of a body susceptible to infection, etc.). Systems/assemblies and methods of the present disclosure can be used with surgical instruments in surgical procedures requiring illumination/treatment. Examples of surgical procedures that may employ assemblies and methods of the present disclosure include, without limitation, open surgical procedures; laparoscopic procedures; endoscopic procedures, insertion of prostheses, anchors and fixation devices, including rods, plates and cables, trocars, injection ports, pacemakers; and/or procedures benefiting from improved illumination/treatment/prevention of surgical site infections (e.g., illumination, UV disinfection/decontamination especially UV in the UVC wavelength range, other UV treatment, infrared heating and treatment, therapy, IR light delivery, use of all/other wavelengths of light, etc.).

In example embodiments, the phototherapy system can include a combination of an optical head unit and a base which can be utilized for medical applications on bodies of people and animals and/or within bodies of people and animals (e.g., illumination, UV disinfection/decontamination especially UV in the UVC wavelength range, other UV treatment, infrared heating and treatment, therapy, IR light delivery, use of all/other wavelengths of light, etc.). Moreover, it is noted that there are also similar uses of an optical head unit and/or a base of the phototherapy system on inanimate objects in medical facilities, in laboratories of various kinds, in industry and elsewhere. The ability to apply UV or other decontaminating/disinfecting radiation is particularly valuable for these medical and non-medical applications where there is a risk of re-introduction of microbes and other kinds of contaminants. In general, embodiments of the present disclosure can be utilized for the delivery of the full range of light spectra for a range of applications (e.g., illumination, therapy, decontamination, disinfection, etc.).

Referring now to the drawings, like parts are marked throughout the specification and drawings with the same reference numerals, respectively. Drawing figures are not necessarily to scale and in certain views, parts may have been exaggerated for purposes of clarity.

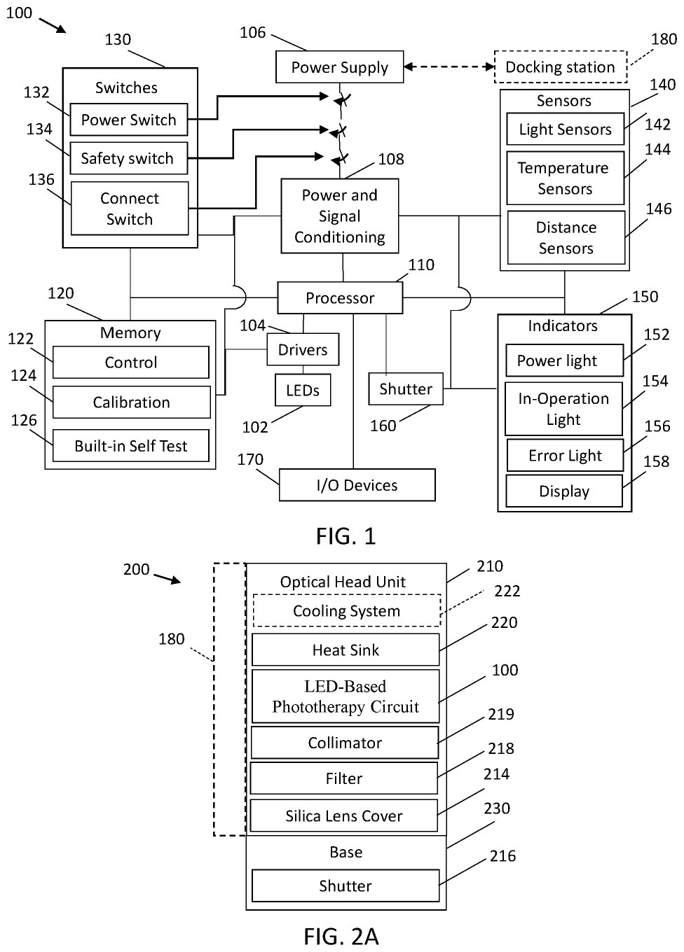

FIG. 1 depicts an example LED-based phototherapy circuit 100 in accordance with embodiments of the present disclosure. The circuit 100 includes light emitting diodes (LEDs) 102, LED drivers 104, a power supply 106, power and conditioning circuitry 108, a processor 110, memory 120, switches 130, sensors 140, and indicators 150. The circuitry 100 can also include a shutter 160. The memory 120 and the processor 110 can be stand-alone separately packaged components or can be packaged or integrated together with or without additional circuitry to form a microcontroller. The circuit 100 can operate to selectively energize the LEDs 102 to emit light at one or more wavelength in the light spectrum, which includes infrared, visible, and ultraviolet light/radiation, to provide illumination, therapy, decontamination , disinfection of a target site. As an example, the circuit 100 can energize the one or more of the LEDs to emit therapeutic light, for example at one or more wavelengths in the ultraviolet (UV) or ultraviolet C (UVC) spectrum, to provide antisepsis phototherapy for infection prevention and treatment, including prevention/treatment of antibiotic resistant infections. The circuitry 100 can operate to output therapeutic light with one or more intensities for a specified time period that is clinically effective in preventing and/or treating infections including, inter alia, bacterial infections (including antibiotic-resistant bacterial infections), viral infections and fungal infections without causing potential long term DNA damage to otherwise healthy tissue upon which the light is incident.

The LEDs 102 can include one or more LEDS configured to emit light in the light spectrum, including infrared, visible, and ultraviolet light spectrums at one or more wavelengths and/or one or more intensities. As a non-limiting example, the LEDs 102 can emit light at one or more wavelengths in a range of approximately 100 nm to approximately 10,000 nm; approximately 100 nm to approximately 400 nm; approximately 400 nm to approximately 700 nm; or approximately 700 nm to approximately 1000 nm. The LEDs 102 can be controlled to output light as specific wavelengths that have been found to be beneficial for decontamination/disinfection of target sites. As a non-limiting example, the LEDs 102 emit light in the UV spectrum in a wavelength range of approximately 207 nm to approximately 265 nm or can emit light at a wavelength of approximately 222 nm, 235 nm, and/or 265 nm. Using light at a wavelength of approximately 235 nm can provide a favorable pathogen inactivation to risk ratio with a high degree of antisepsis effectiveness. The LEDs 102 provide an efficient light source with minimal heat generation, excellent safety profile, longevity, do not require a warm up period, and can be massed produced (for commercialization). One example of LEDs that can be utilized to output light at a wavelength of 235 nm are proprietary LEDs developed and manufactured by Crystal IS, Green Island, NY a division of Asahi Kasei, Japan. Using LEDs that emit light at a wavelength in the UVC spectrum and particularly, at 235 nm can result in inactivation of pathogens including bacteria, viruses and fungi by, for example, disrupting the deoxyribonucleic acid (DNA) of pathogens so that the pathogens cannot replicate nor can they maintain homeostasis and/or by interfering with cytoplasmic protein synthesis of bacteria.

A precise spatial arrangement of the LEDs 102 can be used to ensure homogeneous light distribution with a spatial uniform density +/−10%. The circuitry 100 can adjust the output of the LEDs 102 to customize the light emission from the LEDs to achieve a desired uniformity of light distribution. The output of each LED 102 can be directly related to the input current to each LED 102 which can be controlled via the drivers 104 such that one or more of the LEDs 102 can have different outputs from each other. That is, the circuitry 100 can provide variable input currents to the LEDs 102 to control total cumulative/overall output of the LEDs 102 to achieve one or more parameters (e.g., uniformity, intensity, power, luminance, etc.). As an example, the LEDs 102 can be controlled to have different outputs depending on locations of the LEDs 102 in a specified spatial arrangement of the LEDs 102.

In a non-limiting example embodiment for which the LEDs 102 operate at 235 nm, the LEDs 102 can have an irradiance of approximately 0.5 micro-Watts (μW) at approximately 20 mA to approximately 40 mA current, although the irradiance of the LEDs can be greater or less than 0.5 μW and/or the input current can be greater or less than 20 mA. When seven of the LEDs 102 with these non-limiting example characteristics are used (e.g., 0.5 μW and 20 MA), the LEDs 102 can produce a total output of 3.5 μW dispersed over a target site of 44.2 cm². While the foregoing non-limiting example embodiment includes seven LEDs 102, exemplary embodiments of the present disclosure can include more or fewer LEDs 102. As an example, tens or hundreds of the LEDs 102 can be included in the circuit 100. As another example, between 50 and 100 of the LEDs or between 60 and 90 can be included in the circuit 100. The heat produced by the LEDs 102 is not a limiting factor. As a non-limiting example, for an embodiment in which each of the LEDs 102 operate with a constant current of 20 mA and a 6 volt drop, each LED 102 produces approximately 0.12 watts (20 mA×6 v). Therefore, even if 100 of the LEDs are used, the expected heat production in this non-limiting example is the equivalent of a 12-watt convention incandescent light bulb. Additionally, the heat produced by the circuitry is not transmitted to the target site so the heat does not impact the target site. However, the heat produced by the circuitry can impact an operation of the circuitry 100 itself. To mitigate the impact of the heat produced by the circuitry, the circuitry can automatically shut off when a threshold temperature is reached. Additionally, a docking station 180 can be utilized to dissipate the heat produced by the circuitry 100 between uses as described herein

In one non-limiting example embodiment, the LEDs 102 of the circuitry 100 can output a total energy output of approximately 100 micro-Joules per square centimeter (μ-Joules/cm²) to provide. In other non-limiting example embodiments, the LEDs 102 of the circuitry 100 can output a total energy output of greater or less than 100 micro-Joules per square centimeter. The total energy output by the LEDs 102 can be adjustable by the circuitry 100 or can be fixed. For embodiments in which the output energy has been fixed, a total dosage of light (e.g., IR, Visible, UV, or UVC) can be varied by exposure times. Varying the exposure time during clinical trials can be used to determine ideal dosing, where dosages can be determined based on the energy output by the LEDs 102, the duration for which the energy is output, and a distance between the LEDs and the target site. As the power emitted by the LEDs 102 is relatively low per LED (e.g., ˜0.5 μW), in order to reach a therapeutic exposure time (e.g., of less than 5 minutes), a high density array of the LEDs 102 can be used to achieve a total energy deposited on target surface for a specified exposure time (e.g., a total energy of 100 μJ/cm² or a total energy of greater or less than 100 micro-Joules per square centimeter).

The LED drivers 104 are configured to control an operation of the LEDs 102. The LED drivers 104 can drive the LEDs 102 based on the drive signals received from the processing device 110 to output the light at a specified wavelength and intensity for a specified duration of time. The LED drivers 104 can operate at a constant current (e.g., in one non-limiting example approximately 20 mA) or a variable current to drive a set or string of the LEDs 102 arranged in series with a volt drop (e.g., in one non-limiting example 6 volts) across each of the LEDs 102 in the set or string. In an example embodiment, each set or string of the LEDs 102 can include a specified number of the LEDS (e.g., in a non-limiting example about five to fifteen LEDs or between seven to ten LEDs) so that there can be one driver 104 for each set or string or LEDs. To maintain a constant irradiance, the voltage for each driver 104 can be tuned.

The power supply 106 can be configured to receive power form an external power source and to convert the power to a specified voltage and/or electric current. As one example, the power supply can include one or more transformers, voltage converters (e.g., AC to DC or DC to DC), capacitors, inductors, and/or resistors that operate to convert an input voltage to an output voltage, e.g., for converting a line alternating current (AC) voltage (e.g., 120V AC, 240V AC, etc.) to a specified direct current (DC) voltage. Alternatively, or in addition, the power supply 106 can include an integrated power source that operates to power the circuitry 100 without requiring the circuitry to be connected an external power source during operation/use of the circuitry 100. As an example, the power supply 106 can include one or more batteries. In some embodiments, the circuitry 100 can be powered by a rechargeable battery and can provide for use of an external power source to charge the battery (e.g., via the docking station 180) or power the circuitry as a backup to the battery.

The docking station 180 is generally separate from the circuitry 100 and can be used to charge/re-charge the power supply via an electrical connection to the circuitry 100 or wireless charging. Wireless charging between the docking station 180 and the power supply 106 can include, for example, inductive charging, capacitive charging, RF charging, and/or any other forms of wireless power transfer. The docking station 180 and power supply 106 can include electronic components to facilitate wireless power transfer. As an example, for embodiments that utilize inductive charging/re-charging of the power supply 106, the docking station 180 can include a first induction coil and the power supply 106 can include a second induction coil to charge/re-charge the power supply 106. As an example, for embodiments that utilize RF charging, the docking station 180 can include an RF charging emitter and the power supply can include an RF charging receiver.

The power and conditioning circuitry 108 can receive an output from the power supply 106, condition the voltage and/or current output by the power supply, and distribute the voltage and/or current to the LED drivers 104 (and/or the LEDs 102), the processor 110, the memory 120, the switches 130, the sensors 140, the indicators 150, and the shutter 160. As an example, the power and conditioning circuitry 108 can include one or more voltage regulators, current regulators, filters, and/or electrostatic discharge protection circuits.

The memory 110 can be non-transitory computer-readable media (e.g., computer storage and/or memory, EEPROM, RAM), and can store a control engine 122, a calibration engine 124, and built-in self-test engine 126. The control engine 122 includes executable instructions or code that can be executed by the processor 110 to control an operation of the circuitry 100. The control engine 122 is executed by the processor 110 to control an operation of the circuitry 100 to provide light to a target site at one or more wavelength in the light spectrum including infrared, visible, and ultraviolet light/radiation. For example, the circuitry can provide a dosage of therapeutic light in the UV spectrum. The calibration engine 124 is executed by the processor 110 to calibrate and/or adjust the output of the LEDs 102 and/or the sensors 140 to ensure the circuitry 100 operates as expected. The built-in self-test engine 126 is executed by the processor 110 to perform diagnostic testing on the operation of the circuitry 100 to detect and/or resolve errors in the operation of the circuitry 100. While an example embodiment is shown with the control engine 122, the calibration engine 124, and the built-in self-test engine 126 as separate engines, in exemplary embodiments, the control engine 122, the calibration engine 124, and the built-in self-test engine 126 can be integrated into a single engine to control, calibrate, and test the circuitry 100.

The switches 130 can be operatively coupled between the power supply 106 and the power and conditioning circuitry 108 or can be operatively coupled between a power source and the power supply. The switches 130 can operate to selectively open and close/complete the circuit of the circuitry 100 to control the power to the to the circuitry 100 (e.g., by disconnecting a power source and connecting a power source, respectively). The switches 130 can include a power switch 132, a safety switch 134, and a connect switch 136. The power switch 132 can be the main switch for selectively powering the circuitry 100 and can be a normally open switch that can be closed to energize the circuitry 100 for operation and can be open to de-energize the circuitry 100 based on an interaction between the user and power switch 132 or between the user and a user interface in communication with the power switch 132. The safety switch 134 can be a normally closed switch and can be controlled to open in response to one or more control signals output by the processing device 110 based on an execution of a control engine 122 and/or the built-in self-test engine 126. Alternatively, or in addition, the safety switch 134 can be controlled to open in response to outputs from one or more of the sensors 140. In example embodiments, the circuitry 100 can be included in a housing that is configured to be secured to a base structure. For these example embodiments, the switches 130 can include the connect switch 136, which can be a normally closed switch that can be controlled to open in response the housing of the circuitry 100 and the base structure being decoupled or detached or can be a normally open switch that can be controlled to close in response the housing of the circuitry 100 and the base structure being coupled or attached.

The sensors 140 can sense one or more one or more physical parameters resulting from an operation of the circuitry 100. As an example, the sensors 140 can include light sensor(s) 142, temperature sensor(s) 144, and distance sensor(s) 146. The light sensors 142 can sense an intensity of light being emitted by the LEDs 102. The sensed intensity of the light can be used by the processor 110 to perform self diagnostics, detect errors in the operation of the circuitry, calibrate the LED drivers 104, determine an exposure time, adjust a brightness of one or more of the LEDs 102 via the drivers 104 to improve uniformity in the distribution of energy from the LEDs 102. The temperature sensor(s) 144 can sense temperatures of heat being dissipated by the circuitry 100 to ensure that the circuitry does not operate above a specified threshold temperature. The temperature sensor(s) 144 can also ensure that the circuitry 100 is not operating in a manner that raises a temperature of the bottom of the based above 40° Celsius. The output of the temperature sensor(s) 144 can be input to the processor 110, which in response to determining that the temperature sensed by the temperature sensor(s) 144 exceeds the threshold temperature, can control the safety switch 134 to disconnect power from the circuitry 100. Alternatively, the outputs of the temperature sensor(s) 144 can be operative coupled to the safety switch 134 (e.g., via one or more electrical components other than the processor 110) to control an operation of the safety switch 134. The distance sensor(s) 146 can be used to measure a distance between the LEDs 102 and a target site to be treated by light emitted by the LEDs 102. The distance sensor(s) 146 can be an optical sensor that measure use the transmission and reflection of light to measure the distance. The processor 110 can use the measured distance between the LEDs 102 and the target site to determine an output energy of the LEDs 102 and/or a duration for which the energy is output by the LEDs 102 to administer a specified dosage of light energy to the target site.

The indicators 150 can output one or more parameters associated with an operation of the circuitry 100. The indicators 150 can be controlled by the processor 110 in response to an interaction between a user and the circuitry 100 and/or based on the operation of the circuitry 100 such that the processing device 110 can energize/de-energize or otherwise control one or more of the indicators 150 to correspond to inputs received by the user and/or a state of the circuitry 100. The indicators 150 can be controlled by the processor 110 to represent parameters or values associated with different functions/operations of the circuitry 100. As an example, the indicators 150 can output information about a state of the circuity (“in operation”, power on, power off, errors detected, calibrating, time of exposure, temperature, intensity of light, etc.). The indicators 150 can include a power light 152, an in-operation light 154, an error light 156, and/or a display 158. The power light 152 can be energized when power is being supplied to the circuitry 100 (e.g., when the switches 130 are closed) and can be de-energized when power is not being supplied to the circuitry 100 (e.g., when one of the switches 130 are open). The in-operation light 154 can be energized when the circuitry 100 is outputting light via the LEDs 102 and can be de-energized when the circuitry 100 is not outputting light via the LEDs 102. The error light 156 can be energized when an error is detected in the operation of the circuitry 100 and can be de-energized when no errors in the operation of the circuitry 100 are detected. The display 158 can provide output information regarding the operation of the circuitry 100 and in some embodiments can be provide instead or in addition to the indicator lights 152, 154, and 156. For embodiments in which the display is provide in place of the indicator lights, the display can output indicators regarding the power, operation, and errors of the circuitry 100. In an example embodiment, the display can output a state of the circuitry (e.g., self-testing, calibrating, in-operation, etc.), a sensed temperature, light intensity, a time of exposure (or timer indicating a remaining time of exposure), and the like.

The shutter 160 can be electromechanical device that can control a geometry of an aperture through which the LEDs 102 illuminate a subject. As an example, the shutter 160 can be controlled by the processor 110 to increase or decrease a size of the aperture depending on the area to be irradiated by the LEDs 102. While an example embodiment has been illustrated to include the electromechanical shutter 160, exemplary embodiments may not include the shutter 160 or may include a mechanical shutter that is not controlled by the circuitry 100.

The processor 110 is programmed to execute the control engine 122, the calibration engine 124, and the built-in self-test engine 126 to perform one or more operations. As an example, when power is supplied to the circuitry 100, the processor 110 can execute the built-in self-test engine 126 to perform diagnostic testing of the circuitry 100. If the processor 110 determines that one or more errors in the operation of the circuitry exist (e.g., based on an output of one or more of the sensors 140), the processor 110 can control the indicators 150 to indicate that an error exists. In response to determining an error exists, the processor 110 can prevent the LEDs 102 from further outputting light until the error is resolved. The diagnostic testing performed in response to execution of the built-in self-test engine 126 can include, for example, verification that the LED drivers 104 are operational, and that the housing of the circuitry is secured to the base, and/or that a timer function is operating correctly.

As another example, the processor 110 can execute the calibration engine 124 to calibrate an output of the LEDs 102 (e.g., by controlling an operation of the drivers 104) by increasing or decreasing an input current to the LEDs 102. The processor can use the output of the light sensors 142 as an input during the execution of the calibration engine 124 to calibrate the outputs of the LEDs 102. The processor 110 can execute the calibration engine 124 at the time of manufacture to calibrate the outputs of the LEDs, can execute the calibration engine 124 each time the circuitry turned/powered on, and/or at a specified frequency (e.g., ever fifth time the circuitry is turned/powered on).

In some embodiments, at the time manufacture calibration and testing can be performed using a UV-C camera. For example, the circuitry 100 can be operated to emit light via the LEDs 102 and a digital number read by a UV-C camera detector pixel can be converted to a scientific radiance (watt/steradian/area). The circuitry 100 can be calibrated based on the sensed measurements captured by the UV-C camera detector pixel. The UV-C camera can be calibrated to a NIST standard before testing and calibrating the circuitry 100.

The spatial irradiance homogeneity can depend upon each LED in a string or set contributing an equal amount of radiance to a target area. To achieve this, the optical irradiance of the LEDs 102 in a string or set can be measured via the light sensor 142 or UV-C camera detector pixel during calibration. The current output by the LED driver 104 for the string or set of the LEDs 102 can be increased or decreased to a nominal value selected for each string or set. The nominal value can depend upon the measured LED output power, the UV Fluence of the LED array, and a desired treatment/exposure time. Once each sting or set of LEDs is tuned via adjusting the respective LED driver 104 for each string or set, the entire target area can be observed to ensure that the target area receives a uniform light distribution.

The processor 110 executes the control engine 122 to control a primary operation of the circuitry 100 to emit light at one or more wavelength in the light spectrum, which includes the infrared, visible, and ultraviolet light spectrums. For example, the circuitry 100 operates to emit therapeutic light via the LEDs 102 to provide antisepsis phototherapy for infection prevention and treatment, including prevention/treatment of antibiotic resistant infections. The processor 110 can execute the control engine 122 to output light at one or more specified wavelengths in the ultraviolet (UV) and UVC spectrum, with one or more intensities, for a specified time period that is clinically effective in preventing and/or treating infections including, inter alia, bacterial infections (including antibiotic-resistant bacterial infections), viral infections and fungal infections without causing potential long term DNA damage to otherwise healthy tissue upon which the light is incident. The processor 110 can output one or more drive signals to the one or more LED drivers 104, which can operate in response to the drive signals to control an output of the one or more LEDs 102. The drive signals output by the processor 110 can be pulse width modulated (PWM) signals. The processor 110 can be programmed to execute the control engine 122 to control an intensity of the light output by the LEDs 102 to control the brightness of the light output by the LEDs 102 and/or a duration for which the LEDs 102 output light (e.g., based on a desired dosage and a distance between the LEDs 102 and a target site). As an example, the processor 110 can output the drive signals to the LED drivers 104 based on parameters determined from an execution of the calibration engine 124 and/or a distance between the LEDs 102 and the target site measured by the distance sensor(s) 146 and can control the LED drivers 104 to cause the LEDs 102 to output light for a specified duration of time which can be determined by a timer function executed by the processor 110 using the measured distance. The processor 110 can execute the control engine 122 to operate in different modes depending on the target site. For example, the LEDs 102 can operate at a lower power and/or shorter duration when the target site is skin and the LEDs can operate at a higher power and/or longer duration when the target site is intra-abdominal or open joint applications. The distance between the LEDs 102 and the target site can also be used as an input by the processor 110 to adjust duration for which the LEDs 102 emit light (e.g., the further away the target site, the longer the duration of exposure). The ability to determine a distance to the target site can be important once a body cavity is opened and a new distance to a new target site is calculated because the processor 110 can automatically adjust the output of the LEDs 102 and/or duration of the output of the LEDs when the new distance is detected. The processor 110 can execute the timer function to allow a user to initiate, set, and/or cancel a timer that controls duration for which the LEDs 110 emit light (e.g., a timer can be set for 5 minutes such that after 5 minutes of operation, LEDs can be de-energized). As a non-limiting example, the timer function can be specified so that the processor 110 outputs drive signals to the LED drivers 104 for a specified period of time (e.g., five minutes), after which the processor 110 can cease outputting the drive signals and the LEDs 102 can cease outputting light.

The processor 110 executes the control engine 122 to selectively control which of the LEDs 102 are energized and which of the LEDs 102 are not energized. As an example, the processor 110 can selectively energize a quantity of the LEDs 102 based on one or more parameters or user inputs to, for example, adjust a cumulative intensity/luminance or power being output by the LEDs 102, a total area illuminated by the LEDs 102, and the like.

The processor 110 execute the engines 122, 124, and/or 126 to generate a user interface via the indicators 150 and one or more electrical, electromechanical, mechanical, and/or virtual components. The user interface can allow a user to interact with the circuitry 100 to control an operation of the circuitry 100. In exemplary embodiments, the user interface can include the switches 130, the indicators 150, and other I/O device 170 such as keypads, buttons, dials, selection switches, displays, and the like. In some embodiments, at least a portion of the user interface can be rendered on the display device 158 (which can have a touch screen interface). For example, rather than having physical components such as switches and indicator lights, the display device 158 can render virtual components that can be controlled by the user via an interaction with the touch screen interface. The display device 158 can be integrated into the housing of the phototherapy system and/or can be separate from the circuitry 100 and housing, but in communication with the circuitry via, for example radiofrequency (RF) communication, such as that provided by Wi-Fi, Bluetooth, Zigbee, and/or Z-wave protocols. For embodiments in which the display device 158 communicates with the circuitry via RF communication, the display device 158 and the circuitry 100 can include RF transceivers.

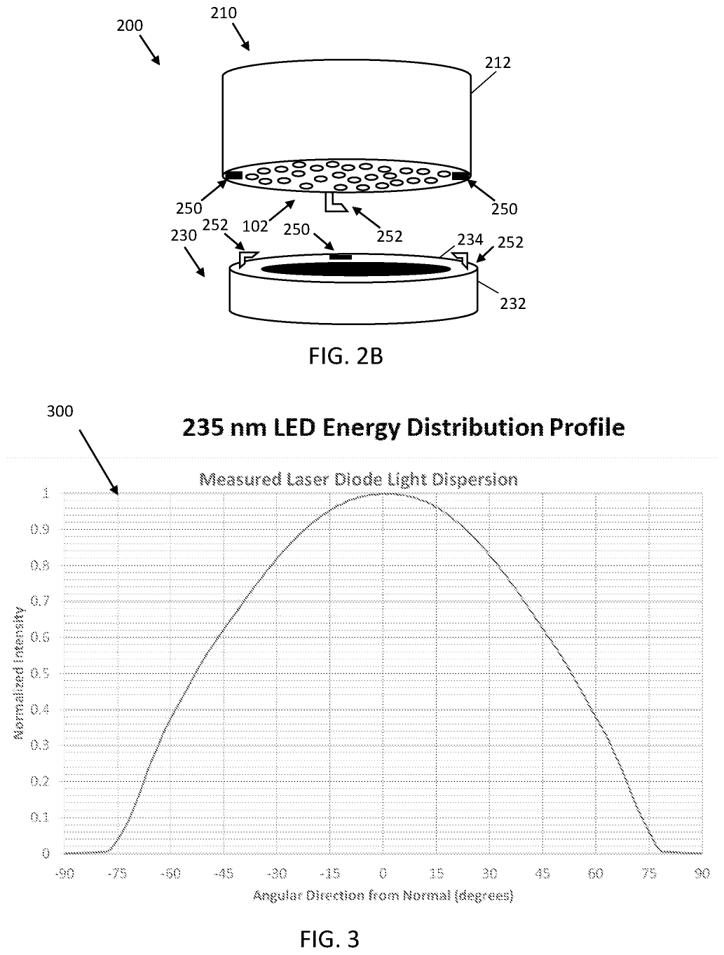

FIGS. 2A-B depict an example phototherapy system 200 in accordance with embodiments of the present disclosure. As shown in FIG. 2, the system 200 can include an optical head unit 210 and a base 230. The system 200 can overcome the shortcomings of other phototherapy delivery platforms including those which are not practical for clinical applications due to size, heat production, lack of safety shielding for clinical personnel, an imprecise dosing mechanism due to variable distance to the treatment target, or an unacceptably high level of DNA mutations following light exposure. The system 200 can deliver a safe dose of phototherapy in under 5 minutes which can result in up to a 90% reduction in colony forming units (CFUs) on the host target site or even up to a 99% reduction.

The optical head unit 210 can incorporate the circuitry 100, a lens cover 214, a collimator 219 and/or filter 218, and a heat sink 220. In some embodiments, the optical head unit 210 can also include a cooling system 222. The LEDs 102 of the circuitry 100 can be disposed along a surface of the optical head unit 210 and can be positioned with respect to the surface in a specified configuration as described herein. The LEDs 102 can be arranged and controlled to provide a homogeneous light distribution pattern within a target area. As an example, the LEDs 102 can be arranged and controlled to generate light having spatial uniformity of approximately 90% and the quantity of LEDs and their placement in the optical head unit can produce an output radiance required to achieve a 90% pathogen inactivation in under 5 minutes or even up to a 99% pathogen inactivation in under 2 minutes. The lens cover 214 can be formed of, for example, silica, and can overlay or cover the LEDs 102 to prevent direct contact with the LEDs 102. The lens 214 can also reflect some of the light emitted by the LEDs 102 and the light sensors 142 of the circuitry 100 can sense the reflected light, which can be used by the processor 110 to estimate the light distribution generated by the LEDs 102. The base 230 may include a shutter 216. The shutter 216 can be a mechanical shutter or an electromechanical shutter (e.g., shutter 160) to adjust an aperture geometry through which the light emitted by the LEDs 102 passes. The shutter 216 can create an aperture with different geometries (e.g., circular, elliptical, rectangular, triangular, etc.) of different sizes, e.g., based on the target area to be illuminated by the LEDs of the Optical head unit 210. The collimator 219 and/or filter 218 can modify the light as it passes through the collimator 219 and/or filter to achieve a desired effect on the light. For example, the collimator 219 may narrow, focus, or control the emission of light to be concentrated on target areas and the filter 218 may be used to filter, block, or reduce certain wavelengths of light which may be harmful to the host tissue.

In an exemplary embodiment the optical housing unit 210 may be sealed, unsealed, and resealed from the outside to protect the internal components from contamination, e.g., bodily fluids or liquids. Further the circuitry 100 and the components housed in the optical head unit 210 may be removed from the optical head unit 210 separately or in an intact assembly such that the circuitry 100 and/or components can be moved between different optical housing units 210, thereby rendering the optical housing unit 210 disposable and potentially obviating the need to sterilize or facilitating the sterilization process of the optical head unit 210.

In an exemplary embodiment, the filter 218 may be an ultraviolet bandpass (UV BP) filter made of a UV fused silica substrate. The filter 218 may be a coating applied to the lens 214 or a standalone filter. The filter 218 may narrow the spectral emissions, filtering light having a wavelength of greater than 240 nm, as light of greater wavelengths, e.g., greater than 240 nm, may cause mutations in the host tissue. However, it is understood that the filter 218 may be made of a different material and/or have varying specification, e.g., thickness, polarization, translucence, angle of incidence to the LED, among others, in order to filter or block any wavelength of light. By way of non-limiting examples the filter 218 may block light outside the range of 100 nm to approximately 10,000 nm; outside the range of approximately 100 nm to approximately 400 nm; outside the range of approximately 400 nm to approximately 700 nm; outside the range of approximately 700 nm to approximately 1000 nm; greater than 235 nm; greater than 254 nm; greater than 265 nm; less than 207 nm and greater than 222 nm; although not limited thereto. In an embodiment the collimator 219 and/or filter 218 may be dynamic whereby the wavelength of the filter light is changed in real time. The collimator 219 and/or filter 218 may be used to vary and adjust the wavelength, intensity, and/or concentration of the light that ultimately passes through the lens 214 and is emitted by the phototherapy system. In this way the collimator 219 and/or filter 218 may be used in place of or to supplement a controller which selectively energizes the LEDs to emit light at one or more wavelength in the light spectrum, e.g., similar to how circuit 100 operates LEDs 102.

In an exemplary embodiment, a housing 212 of the optical head unit 210 can be general cylindrically shaped, having a specified diameter and thickness. As a non-limiting example, the housing 212 can have a diameter that is approximately two to ten inches (e.g., in a non-limiting example embodiments the diameters can be 3, 5, and 8 inches) and can have a thickness that is approximately a half inch to approximately five inches. The array of the LEDs 102 and quantity of the LEDs 102 included in the circuitry can scale based on a diameter of the housing (e.g., as the diameter increases the quantity of the LEDs 102 in the circuitry 100 can increase). The heat sink 220 and cooling system 222 (e.g., a fan) can aid in removing heat dissipated by the circuitry 100.

The base 230 can be selectively coupled to the optical head unit 210. As an example, the optical head unit 210 can engage and be secured to the base 230 during use of the system 200 can be disengaged from the base 230 when the system 200 is not in use. The base 230 can operate as a light shield to prevent light emitted by the LEDs 102 from illuminating unintended areas (e.g., to protect against undesired exposure to the light emitted by the LEDs 102) and provides a spacer or cavity that provides for a standardized exposure distance for the light being emitted by the LEDs 102 (measureable by the distance sensor(s) 146). In some embodiments, the base 230 can be disposable or reusable. Inadvertent exposure to light from the LEDs 102 can be avoided by using a safety interlock between the optical head unit 210 and the base which can rest around a target site. All emissions from the LEDs 102 in the optical head unit 210 can be directed directly to the target site. Stray light (unintended light that finds its way out base) can be eliminated through the tight seal and interlock mechanism between optical head unit 210 and the base 230. The base 230 may also provide a standard uniform distance from the optical head unit 210 to the target site. A standard distance from the optical head unit 210 to the target site may provide a baseline for intensity of the LEDs 102 and spectral emissions and may ensure that phototherapy dosing is predictable and consistent. During use, gel pads can also be located on the bottom of the base 230 to allow it to conform to contours of the target site (e.g., contours of a human or animal body), thereby compensating for the irregularities in the contours of the target site increasing uniformity of the distance from the optical head unit 210 to the target site.

In some embodiments, the base 230 can be configured to receive one or inserts that change a size or geometry/shape of an aperture through which the light from the LEDs 102 passes. The inserts can be provide in place of or in addition to the shutter 216 to change the aperture.

While FIG. 2A illustrates the system 200 in an exemplary “stack” it is understood that the components thereof need not be arranged in a particular order whereby the certain embodiments will utilize and benefit from the components being arranged differently. For example in an embodiment the filter 218 may be a substrate coating applied to the underside of the lens 214 facing the base 230 or in an alternate embodiment the filter 219 may be a polarized sheet of silica positioned anywhere between the circuitry 100 and the base 230. These embodiments are provided by way of example, are non-limiting, and are not exhaustive of the order of the components which are contemplated herein.

As show in FIG. 2B, an embodiment of the optical head unit 210 can include a housing 212 having a cylindrical shape and the base 230 can include a housing 232 having a ring or annular configuration. The LEDs 102 can be distributed on one side of the housing 212, which is configured to face and engage the housing 232 of base 230 to position the LEDs 102 within a perimeter 234 of an opening in the housing 232. The housing 212 of the optical head unit 210 can be selectively secured to the housing 232 of the base 230. As an example, the housing 232 can include mating members 250 and the housing 232 can include corresponding mating member 252 that can engage each other to secure the housing 212 to the housing 232. As another example, the housing 232 can include a combination of the mating members 250 and 252, and the housing 232 can include corresponding combination of mating member 252 that can engage each other to secure the housing 212 to the housing 232. In the example embodiment illustrated in FIG. 2B, the mating members 250 are slots and the mating members 252 are projections or locking tabs that are configured and dimensioned to be received by and secured in the slots.

The system 200 provides for appropriate phototherapy dosing based on a distance between the LEDs and the target site in which the distance to the target site can be fixed via the base 230 and/or measureable via the distance sensor(s) 146. The irradiance output by the LEDs 102 and/or the exposure time can be controlled by the circuit 100 based on the distance between the LEDs and the target site. The distance from LEDs to target site, for surface treatments, can in some instances be determined based on the height of housing 232 of base 230 as LEDs are almost flush with top of housing 232 of the base 230. For example, if the height of the base ring is 25 mm, the distance between the LEDs and the surface treatments can be assumed to be 25 mm and/or can be measured via the distance sensor(s) 146. For phototherapy treatment within body cavities, the distance to the target site will be further than surface target and measureable by the distance sensor(s) 146, where the measured distance is used to determine LED output and/or duration of phototherapy treatment to provide appropriate treatment dosage. The light sensor(s) 142 of the circuitry 100 can confirm appropriate dosing has been delivered. The system 200 can be integrated into a surgical setting without significant disruptions to the surgical process. Medical personnel and the patient's other body parts can be protected from inadvertent light exposure by the base 230 which can form a tight interlocked seal with the optical head unit 210. An operating temperature of a bottom of the base can be maintained below 40° C. Containment of phototherapy is also accomplished by flexible pads on the undersurface of the housing 232 of the base 230. This allows for the base ring to conform to body cavities. The thickness of these pads is incorporated into the total height of the housing 232 of the base 230.

FIG. 3 is a graph 300 illustrating an illumination profile of an example LED emitting light at a wavelength of 235 nanometers in accordance with embodiments of the present disclosure. The energy profile of the can be used to estimate/calculate a performance of different quantities of the LEDs 102 working in concert with each other. To achieve a total energy output of 300 μJ/cm2, the system 200 can incorporated the illumination profile for the 235 nm LED into an irradiance profile. This profile can be used to develop an LED Spatial illumination model (LIS) for determining the energy fluence at a spatial field point (e.g., the surgical site to be disinfected).

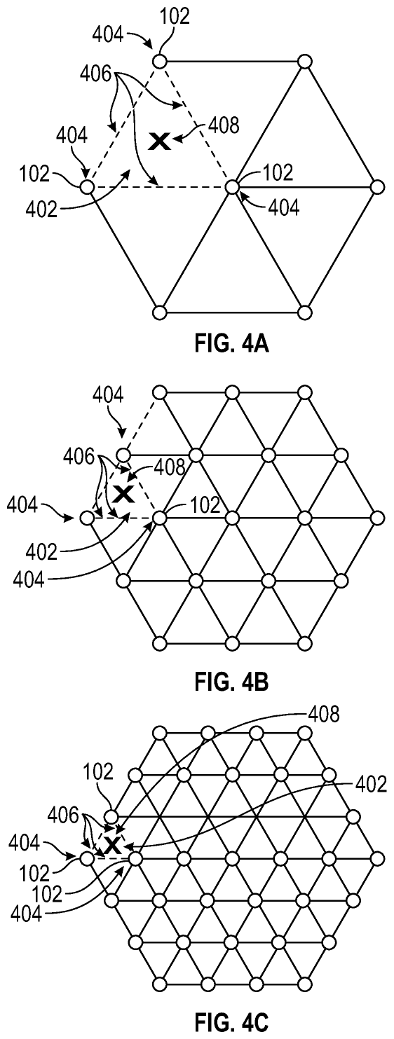

FIGS. 4A-C depict example LED arrangements in accordance with embodiments of the present disclosure. With reference to FIGS. 4A-C, to minimize spatial variations in the field, the LEDs 102 can be arranged as triangular unit cells 402 with the LEDs 102 located at the vertices 404 of each side of the triangle unit cells as described herein. The dimension of the triangle unit cells (side length 406) can be specified so that the illumination in the geometric center 408 of the triangle unit cells 402 at the surgical site can be identical to illumination at each vertex 404. The arrangement of the LEDs 102 can also be described as a lattice structure having a hexagonal perimeter. As shown in FIG. 4A, six triangles unit-cells can be placed together to form a hexagon, which can provide a basic geometry for the LEDs 102. The area of illumination can be increased by adding rows of triangle unit-cells around each side of the original hexagon. As one example, a basic hexagonal form includes seven (7) of the LEDs 102 with 28 mm spacing between the LEDs 102. The density of the LEDs 102 can be adjusted to increase or decreases the energy output per area (e.g., cm²). For example, the size of the triangle unit cells (length of the sides of the triangle can be increased to reduce the energy output per area or can be decreased to increase the energy output per area. The packing density of the LEDs 102 and quantity of the LEDs 102 can be specified so that the LEDs 102 output 300 μJ/cm2 within 5 minutes of operation. As an example, FIG. 4B shows an arrangement of LEDs 102 including 24 triangle unit cells with the same area defined by the arrangement shown in FIG. 4A such that the density of the LEDs 102 in FIG. 4B increased by approximately 4× as compared to the density of the LEDs 102 in FIG. 4A. As another example, FIG. 4C shows an arrangement of LEDs 102 including 54 triangle unit cells with the same area defined by the arrangement shown in FIGS. 4A and 4B such that the density of the LEDs 102 in FIG. 4B increased by approximately 9× as compared to the density of the LEDs 102 in FIG. 4A and approximately 2× as compared to the density of the LEDs 102 in FIG. 4B. While the example embodiment illustrated in FIGS. 4A-C illustrate lattice arrangement of the LEDs 102 with a hexagonal perimeter, embodiments of the present disclosure can have different arrangement of the LEDs 102 with differently shaped perimeters. As an example, in embodiments of the present disclosure, the LEDs can be arranged in concentric circles, squares, rectangles; linear rows and columns; or other arrangements to provide a generally uniform dosage of light to a target site and/or can have circular perimeter, rectangular perimeter, triangular perimeter, trapezoidal perimeter, hexagonal perimeter, or a differently shaped perimeter.

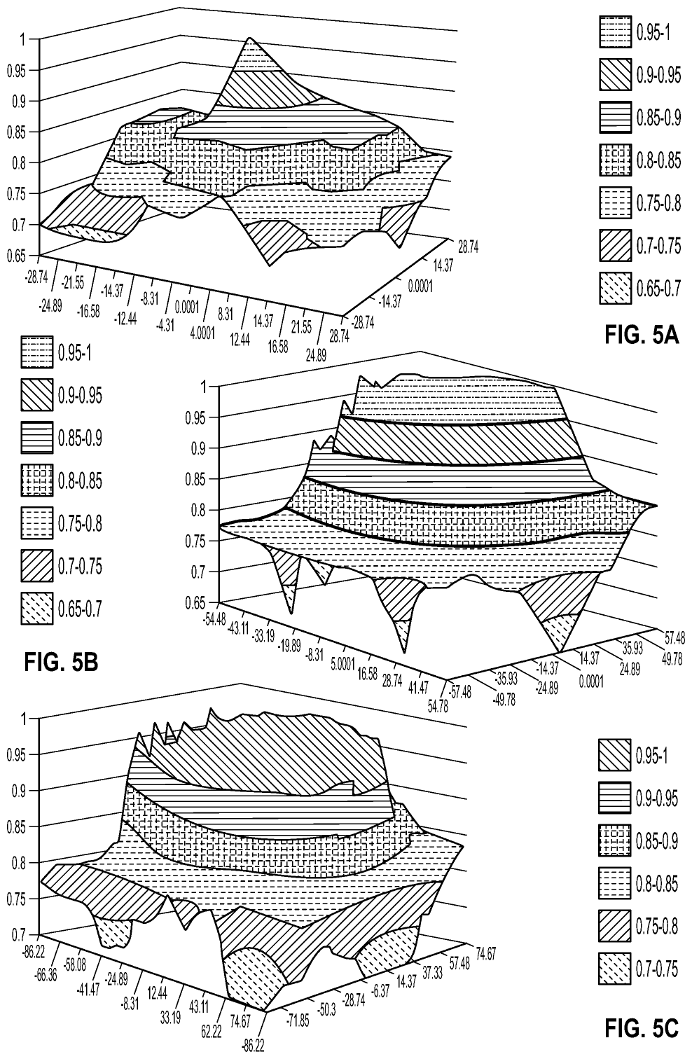

FIGS. 5A-C depict example simulated energy profiles for the LED arrangement of FIGS. 4A-C, respectively, in accordance with embodiments of the present disclosure. As shown in the energy profiles of FIGS. 5A-C, the uniformity of the energy profile improves as the number of triangle unit cells 402, and therefore LEDs 102, increases. The symmetrical non-uniformities in the profiles are model artifacts corresponding to limitations in sampling density and capabilities of the 3-D surface plotting package used to simulate the profiles.

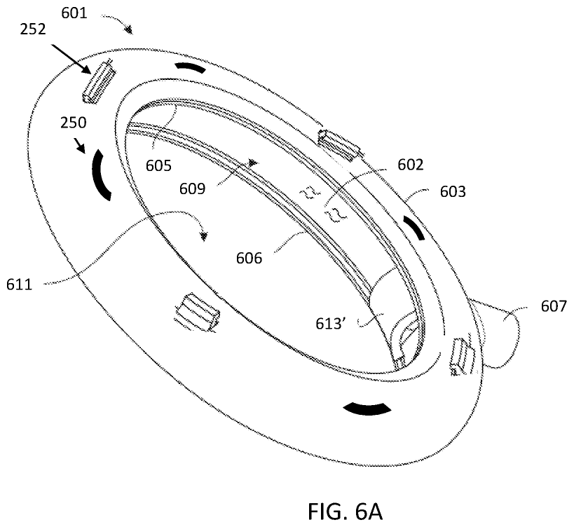

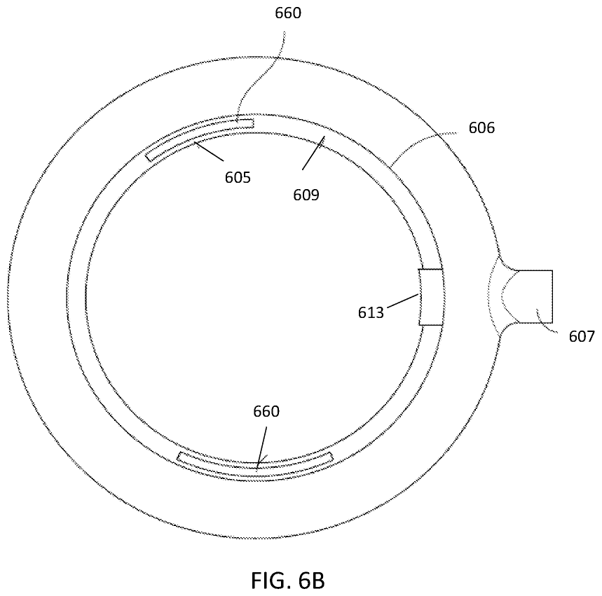

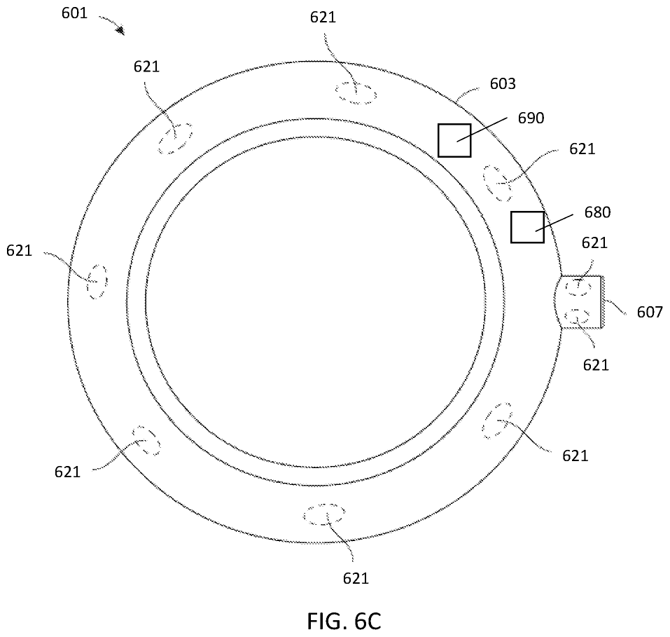

With reference to FIGS. 6A-C, there is illustrated an example embodiment of the base 230, which can be an exemplary phototherapy assembly 601, according to the present disclosure, such that embodiments of the optical head unit 210 and the assembly 601 can be used in combination to form an embodiment of the LED-based phototherapy system 200 that can illuminate an area with ultraviolet light and can also operate to illuminate an area with visible light. The combination of the assembly 601 and/or the optical head unit 210 can be utilized for medical applications on bodies of people and animals and within bodies of people and animals (e.g., illumination, UV disinfection/decontamination especially UV in the UVC wavelength range, other UV treatment, infrared heating and treatment, therapy, IR light delivery, use of all/other wavelengths of light, etc.). Moreover, it is noted that there are also similar uses of assembly 601 and the optical head unit 210 on inanimate objects in medical facilities, in laboratories of various kinds, in industry and elsewhere. For example, use of the LED-based phototherapy system 200 can be used to apply UV or other decontamination and/or disinfecting radiation for medical and non-medical applications where there is a risk of re-introduction of microbes and other kinds of contaminants In general, assembly the LED-based phototherapy system based on the combination of an embodiment of the optical head unit 210 and an embodiment of the assembly 601 can be utilized for the delivery of the full range of light spectra for a range of applications (e.g., illumination, therapy, decontamination, disinfection, etc.).

FIG. 6A gives a perspective view of assembly 601 showing shell 603 and connection feature/coupling 607. Reflector 613′ (or 613 shown in FIG. 6B) can be mounted substantially within shell 603. Shell 603, forms a ring (light ring) having a cross-sectional shape substantially like an asymmetrical C with an upper portion 605 overhanging a lower portion 606. Stated another way, exemplary shell 603 has an asymmetrical C-shaped cross-sectional profile, with the shell 603 having an upper portion 605 and a lower portion 606 with the upper portion 605 extending over the lower portion 606 and extending further inwards than the lower portion 606 to define the asymmetrical C-shaped cross-sectional profile of the shell 603. The reflector 613 can include a faceted surface (e.g., multi-faceted surface) that is angled towards incoming light arriving through connection feature 607. The reflector 613 can have a pyramidal aspect, other embodiments are within the scope of the present disclosure. In some embodiments, reflector 613 can have two facets. The facets of reflector 613 may be flat, curved, or irregular. Reflector 613 can include any suitable number of facets. Due to the fact that exemplary faceted surface presents a plurality of surfaces angled away from one another, incoming light is reflected in a plurality of directions into shell 603. Within shell 603, the light is reflected and re-reflected throughout the shell 603 while also being reflected out of the shell 603 in an inward and downward direction (e.g., towards the pre-determined location or site). The assembly 601 can also include an embodiment of mating members 252 for securing the assembly to embodiments of the optical head unit 210 as described herein.

While FIG. 6A, for example, shows shell 603 being substantially circular in overall shape, it is appreciated that other shapes are possible and can also be adapted to reflect light inwardly and downwardly. For example, a closed curve or polygonal shape can perform as described, as will shapes with a plurality of arms radiating away from reflector 613, such as a V shape or U shape with reflector 613 and connection feature 607 at a point such as the base of the U or V, along one of the arms, or at one or both of the tips of the U or the V. Additional, the shell 603 can be formed as a single integrated structure or can be formed a two or more structures that can be secured together.

In certain embodiments and as shown in FIG. 6A, assembly 601 can include an optional window/lens member 602 (e.g., transparent or translucent window member 602) positioned between the light ring of shell 603 and the site to be illuminated/treated. In general, the window member 602 provides a physical barrier (to fluids/gases, etc.) between the reflective surface 609 of the shell 603 and the site/patient, and protects the reflective surface 609 from contamination (while letting light to pass through window 602). In certain aspects, the window member 602 can modify light intensity and/or wavelength to achieve a preferred pattern of light distribution. In some embodiments and as shown in FIG. 6A, window member 602 can extend from upper portion 605 to lower portion 606 and around/above the inner surface 609 to prevent fluids/gases and the like from contacting inner surface 609, while letting light from inner surface 609/shell 603 to pass through window 602 and to the site.

In some embodiments, the window member 602 provides an inner shell/barrier that diffuses light to improve the quality of the light (e.g., for a surgeon), and/or encloses portions of the assembly 601 to prevent contamination (e.g., to keep fluids out of the light ring/inner surface 609). As such, the assembly 601 can include a transparent or translucent window member 602 that effectively encloses the light ring by covering the C-shaped portion of the light ring. The window member 602 separates the reflective surface 609 of the light ring/shell 603 from fluids (e.g., body fluids of the patient/user) by providing a physical barrier. In other aspects, the window member 602 can modify light intensity and/or wavelength to achieve a preferred pattern of light distribution.

It is noted that light source 717 (e.g., light box) can include one or more LEDs that generate the light (e.g., visible or UV or UVC or other wavelengths of light) for assembly 101. As such, LEDs associated with source 717 and external to assembly 601 can generate the light, with the light being provided to assembly 101 via cable(s) 709 (e.g., via fiber optic tube(s) or cable(s)).

Moreover, it is noted that in addition to or in lieu of light being provided to assembly 601 via light source (via external light source 717), light (e.g., visible or UV or UVC or other wavelengths of light) can be provided to assembly 601 (to shell 603) via one or more LEDs 621 positioned/mounted with respect to coupling 607, as shown in FIG. 6C. For example, the interior of coupling 607 can include one or more LEDs 621 that provide light for assembly 601 (FIG. 6C). It is noted that coupling 607 can include and/or be associated with any suitable number of LEDs 621.

Furthermore, it is noted that in addition to or in lieu of light being provided to assembly 601 via light source 717 and/or via LEDs of coupling 607, light can be provided to assembly 601 (to shell 603) via one or more LEDs 621 positioned/mounted with respect to coupling shell 603 (e.g., to inner surface 609), as shown in FIG. 6C. For example, the inner surface 609 of shell 603 can include one or more LEDs 621 distributed/mounted thereon that provide light for assembly 601 (FIG. 6C). It is noted that shell 603/inner surface 609 can include and/or be associated with any suitable number of LEDs 621. The LEDs 621 can be operatively coupled to an power source 680 (e.g., one or more batteries) integrated in the shell 603 via one or more LED drivers 690. For embodiments of the light assembly 601 that include the integrated power source 680, the light assembly 601 may not include the coupling 607. The light emitted by the LEDs can have wavelengths in the visible light spectrum and/or other spectrums, such UV or UVC, infrared, near infrared, and the like. As a non-limiting example, the light emitted by the LEDs 621 have be in a wavelength range of approximately 400 nanometers to approximately 780 nanometers.

Providing assembly 601 with circular or other open-format morphology of shell 603 allows a user/surgeon to work freely in the area surrounded by assembly 601. In some embodiments, an outer diameter of assembly 601 is between about 2 inches and about 10 inches. In certain embodiments, an outer diameter of assembly 601 is between 4 and 8 inches, or about 5 to about 7 inches. Moreover, assembly 601 can be spaced away from the incision or from surgical instruments. Surgical light is discussed in U.S. Pat. Nos. 7,909,761; 5,488,696; and 4,605,990, the contents of each of which are incorporated by reference in their entireties.

In some embodiments, assembly 601 is provided with an adhesive, such as a peel-and-stick adhesive on one surface.

With reference to FIGS. 6A-6B, a cover member 615 can include one or more reflectors 617. In general, reflectors 617 are mounted with respect to the lower surface 118 of cover member 115. In exemplary embodiments, each reflector 117 is configured and dimensioned to direct light from the assembly 601 (e.g., from shell 603) onto a surface (e.g., the patient or other surface) to achieve a substantially uniform light distribution or any other light distribution that may be desired. It is noted that in addition to or in lieu of reflectors 617, lower surface 618 can include a reflective material/surface or the like, as similarly noted with inner surface 609 (e.g., includes a coating of a reflective material, and/or include mirrors or the like).

Furthermore and also as shown in FIG. 6B, cover member 115 can include one or more LEDs 621. In general, LEDs 621 are mounted with respect to the lower surface 618 of cover member 615. It is noted that cover member 615 can include any combination or permutation of reflectors 617 and/or LEDs 621 (e.g., only reflectors 617, only LEDs 621, both reflectors 617 and LEDs 621, no reflectors 617 and no LEDs 621, etc.). In exemplary embodiments, each LED 621 is configured and dimensioned to generate light (in any wavelength) and direct the light onto the patient or other surface to achieve a substantially uniform light distribution in a given wavelength or any other light distribution that may be desired.

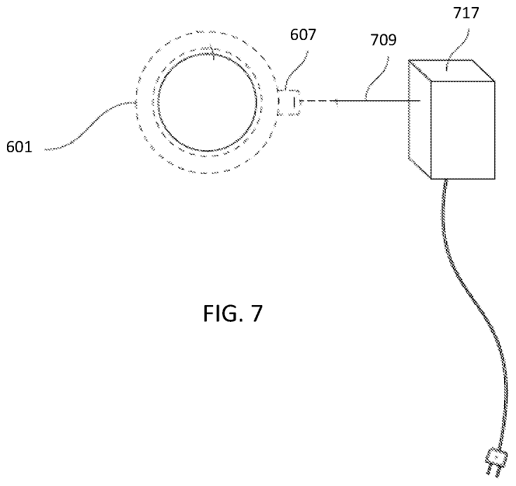

FIG. 7 illustrates use of exemplary assembly 601 according to certain embodiments. Assembly 601 can be connected to light source 717 via cable (e.g., fiber optic cable) 709. Assembly 601 includes connection feature 607 which can include an adaptor to couple to cable 709 (e.g., fiber optic cable or fiber optic light line) from light source 717. The light source 717 may include glass fiber optic cables, plastic fiber optic cables or any other suitable means for transmitting and emitting light. Light source 717 may be any suitable device for producing light such as, for example, a halogen or incandescent light box or other light source 717 readily available in hospital settings. Suitable light sources 717 are available from Welch Allyn Inc. (Skaneateles Falls, N.Y.). The light source 717 may have any suitable power level. Other types of light sources 717 include, without limitation, ultraviolet (UV) light sources, UVC light sources, light-emitting diodes (LEDs), Xenon, Br-KR, Mercury, visible light, IR light sources, other wavelengths of light, etc.

In certain embodiments, light source 717 is the XLS-300 High-Powered 300 W xenon light source from Olympus Corporation (Shinjuku, Tokyo, JP). Any other suitable light source 717 capable of producing light that is transmitted via the light transmitters, such as fiber optic cables, may also be used. Light sources and optic cables are discussed in U.S. Pat. Nos. 5,850,496 and 5,115,126, and surgical illumination generally is discussed in U.S. Pat. Nos. 5,785,648; 6,616,603; 7,150,714; and 5,353,786, the contents of each of which are incorporated by reference in their entireties.

It is noted that light source 717 (e.g., light box) can include one or more LEDs that generate the light (e.g., visible or UV or UVC or other wavelengths of light) for assembly 601. As such, LEDs associated with source 717 and external to assembly 601 can generate the light, with the light being provided to assembly 601 via cable(s) 709 (e.g., via fiber optic tube(s) or cable(s)).

Moreover, it is noted that in addition to or in lieu of light being provided to assembly 601 via light source 717 (via external light source 717), light (e.g., visible or UV or UVC or other wavelengths of light) can be provided to assembly 601 (to shell 603) via one or more LEDs 621 positioned/mounted with respect to coupling 607, as shown in FIG. 6C. For example, the interior of coupling 607 can include one or more LEDs 621 that provide light for assembly 601 (FIG. 6C). It is noted that coupling 607 can include and/or be associated with any suitable number of LEDs 621.

Furthermore, it is noted that in addition to or in lieu of light being provided to assembly 601 via light source 717 and/or via LEDs of coupling 607, light can be provided to assembly 601 (to shell 603) via one or more LEDs 621 positioned/mounted with respect to coupling shell (e.g., to inner surface 609), as shown in FIG. 6C. For example, the inner surface 609 of shell 603 can include one or more LEDs 621 distributed/mounted thereon that provide light for assembly 601 (FIG. 6C). It is noted that shell 603/inner surface 609 can include and/or be associated with any suitable number of LEDs 621.

In other embodiments, the present disclosure provides for an ultraviolet light (or other wavelength range or mixture of wavelengths) delivery system/assembly 601 in an open ring shaped configuration (e.g., to reduce or eliminate contamination of gases in a breathing/ventilator circuit or the like). The light is capable of inactivating bacteria, viruses and fungi or other pathogens. The light may be delivered either continuously or in a pulsed mode.

In one embodiment, the shell 603 (e.g., inner surface 609) is lined with a reflective material capable of reflecting and/or refracting light so the radiation is directed toward the lumen 611 of the shell 603. Light enters the shell 603 from a cable 709 (e.g., fiber optic cable) and is dispersed by a reflecting/refracting lens 602 or lenses 602. In such embodiments, it is noted that assembly 601 may or may not include reflector 613 or the like. The shell 603 can be positioned in-line with the breathing/ventilator circuit or the like (e.g., to reduce or eliminate contamination of gases in the breathing/ventilator circuit or the like). As gases pass through the center of the shell 603 they are exposed to the radiation being directed toward the center of the shell 603. The light is capable of inactivating bacteria, viruses and fungi or other pathogens, and the light may be delivered either continuously or in a pulsed mode.

In other embodiments, the shell 603 is covered on the inside with LEDs 621 (FIG. 6B) that generate ultraviolet light (or other wavelength range or mixture of wavelengths) and direct the light toward the lumen 611 of the shell/ring 603. The shell 603 can be positioned in-line with the breathing/ventilator circuit or the like. As gases pass through the center of the shell 603 they are exposed to the radiation being directed toward the center of the shell 603.

In some embodiments, the shell 603 contains or is associated with (e.g., on the inside surface 609) one or more rigid or flexible fiber optic tubes that generate ultraviolet light (or other wavelength range or mixture of wavelengths) and direct the light toward the lumen 611 of the shell 603. In such embodiments, assembly 601 typically does not include cover member 615. The shell 603 can be positioned in-line with the breathing/ventilator circuit or the like. As gases pass through the center of the shell 603 they are exposed to the radiation being directed toward the center of the shell 603.

In addition to generating and delivering light by the assemblies/structures discussed above, it is noted that visible, UV or other wavelengths of light may be generated and delivered by a rigid or flexible fiber optic tube that delivers light along its length either in all directions (e.g., like a tubular fluorescent light bulb), or in specific directions along its length.

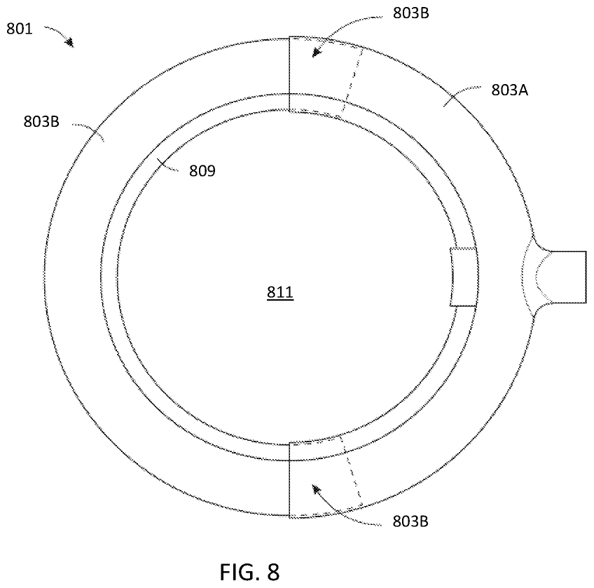

In other embodiments and as shown in FIG. 8, exemplary embodiment of the base 230 embodiments as phototherapy assembly 801, which is similar to assembly 601 discussed above, with some differences. Similar to assembly 601 discussed above, phototherapy assembly 801 is configured and dimensioned to provide light to and/or illuminate/treat predetermined locations (e.g., surgical sites).

Exemplary assembly 801 includes shell member 803A and shell member 803B that are configured to mount with respect to one another to define opening 811, and to define inner surface 809 (e.g., a surface similar to surface 609).

In general, shell members 803A and 803B are adjustable/movable relative to one another so that a user can change the size of opening 911 defined by mounted shell members 803A, 803B. It is also noted that shell member 803B can be removed from shell member 803A, and shell member 803A can then be used without member 803B to provide light to and/or illuminate/ treat predetermined locations (or vice versa).

In certain alternative embodiments, assemblies and methods of the present disclosure incorporate multiple structures (e.g., 601) to provide light both above and below the incision site. Dual-device structures are provided, for example, for use with single incision laparoscopic surgery procedures (SILS). Such procedures may employ an SILS port. See, e.g., U.S. Pub. No. 2012/0130186; U.S. Pub. No. 2012/0022333; U.S. Pub. No. 2011/0021877; and U.S. Pub. No. 2010/0249523, the contents of each of which are incorporated by reference in their entireties.

Two of assembly 601 that are permanently attached one on top of the other can form a multi-structure assembly/system according to an alternative embodiment. The light port lies at the junction of the two light rings and is anchored in place during the manufacturing process to the upper lip of the lower ring. This forms a seal that prevents the escape of gasses from the body cavity during the laparoscopic procedure. The shell 603 of each assembly 601 can be constructed from non-translucent material (e.g., plastic) and has a reflective inner surface 609.

In certain embodiments, the upper one of assembly 601 has two light adaptors. The first adaptor is in a longitudinal plane as above. The second light adaptor is oriented in a vertical plane and is used to provide light to the lower light ring. A direct connection between upper and lower assemblies 601 is aligned and forms a channel to allow light from the second adaptor to pass through the upper assembly 601 to the lower assembly 601. The light then encounters the light reflector, which will reflect light around the lower assembly 601. Light is then directed by the reflective surface in an inferior direction thereby illuminating the body cavity and the in vivo surgical site. An inflatable seal located on the outer diameter of the upper assembly 601 may be provided to prevent gasses from escaping the body cavity. Single-incision procedures are discussed in U.S. Pub. No. 2012/0116362 and U.S. Pub. No. 2008/0064931, the contents of which are incorporated by reference in their entireties.

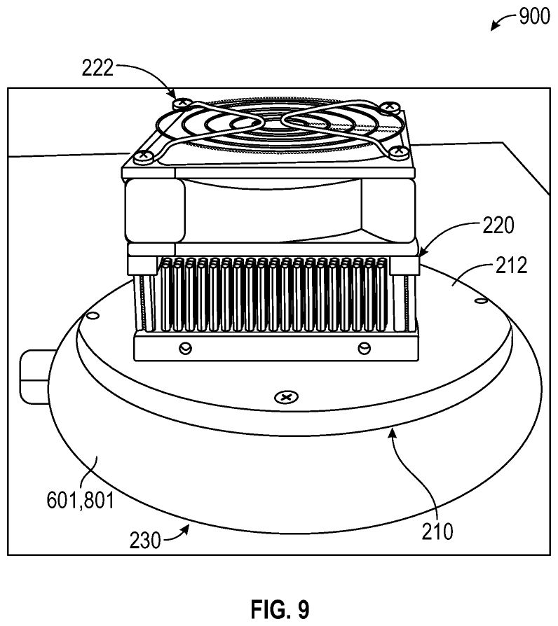

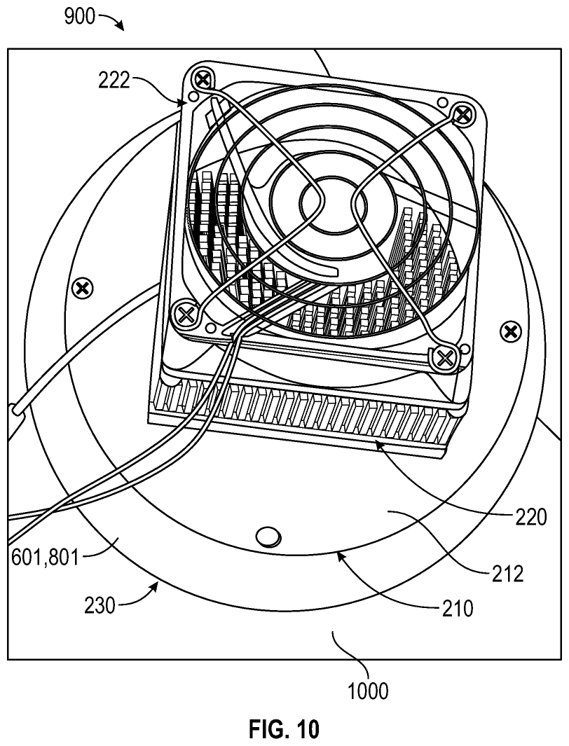

FIGS. 9 and 10 illustrate an experimental embodiment of the LED-based phototherapy system 900 that includes an embodiment of the optical head unit 210 with the housing 212, the heat sink 220, and the cooling system 222 (e.g., a fan) in combination with an embodiment of the base 230 (embodied as the assembly 601 or 801). The optical head unit 210 is securely attached to the base 230, which can be disposed on a surface 1100 (e.g., a body of an animal) to be illuminated by the system 900, as shown in FIG. 10.

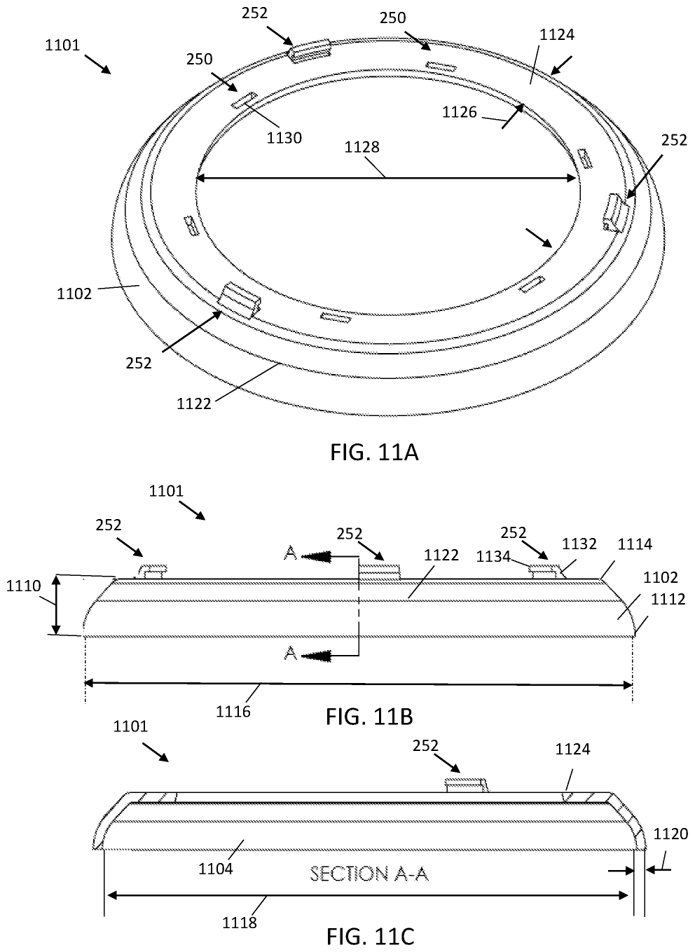

With reference to FIGS. 11A-C, there is illustrated an example embodiment of the base 230, which can be an exemplary phototherapy assembly 1101, according to the present disclosure, such that embodiments of the optical head unit 210 and the assembly 1101 can be used in combination to form an embodiment of the LED-based phototherapy system 200 that can illuminate an area with ultraviolet light and can also operate to illuminate an area with visible light. The combination of the assembly 1101 and/or embodiments of the optical head unit 210 can be utilized for medical applications on bodies of people and animals and within bodies of people and animals (e.g., illumination, UV disinfection/decontamination especially UV in the UVC wavelength range, other UV treatment, infrared heating and treatment, therapy, IR light delivery, use of all/other wavelengths of light, etc.). Moreover, it is noted that there are also similar uses of assembly 1101 and embodiments of the optical head unit 210 on inanimate objects in medical facilities, in laboratories of various kinds, in industry and elsewhere. For example, use of the LED-based phototherapy system 200 can be used to apply UV or other decontamination/disinfecting radiation for medical and non-medical applications where there is a risk of re-introduction of microbes and other kinds of contaminants. In general, assembly the LED-based phototherapy system based on the combination of an embodiment of the optical head unit 210 and an embodiment of the assembly 1101 can be utilized for the delivery of the full range of light spectra for a range of applications (e.g., illumination, therapy, decontamination, disinfection, etc.).

The assembly 1101 can have a general shape of a truncated cone having an outer surface 1102 and an inner surface 1104. The assembly 1101 can have a height 1110 (excluding the mating members 252) measured from a bottom 1112 of the assembly 1101 to a top of the assembly 1114, an outer diameter 1116 and an inner diameter 1118 that generally decrease along the height 1110 of the assembly 1101 from a bottom 1116 of the assembly 1101 to a top of the assembly 1118, and a thickness 1120. The profile of the assembly 1101 can have two sections. The inner diameter 1118 at the bottom 1112 of the assembly 1101 can define a bottom opening of the assembly. A first section can extend from the bottom 1112 of the assembly 1101 to a transition 1122 and a second section can extend from the transition 1122 to the top 1114 of the assembly 1101. The first section can taper inwardly towards a center axis of the assembly 1101 with a first curvature from the bottom 1112 to the transition 1122 and the second section can taper inwardly towards the center axis of the assembly 1101 with a second curvature along the height 1110 from the transition 1122 to the top 1114. The first and second curvatures can be the same or different. While an example embodiment of the assembly includes first and second sections, embodiments of the assembly 1101 can taper linearly or with a uniform curvature along the height from the bottom 1112 to the top 1114.

The top 1114 of the assembly 1101 can include an annular or ring-shaped mounting surface 1124 upon which embodiments of the optical head unit 210 can be mounted. The mount surface can have a width 1126 measured radially with respect to the center axis of the assembly 1101 and can define a top opening 1128 such that the top opening 1128 is smaller in diameter than the bottom opening. The mounting surface 1124 can include the mating members 250 and 252, which can be embodied as slots 1130 and locking tabs 1132, respectively, and can engage corresponding ones of the mating members 250 and 252 on embodiments of the base 230. The locking tabs 1132 can extend upwardly from the mounting surface 1124. The locking tabs 1130 can include lip 1134. At least a porting of the locking tabs 1132 including the lip 1134 can be received in and secured to corresponding mating members 250 on embodiments of the optical head unit 210. The bottom 1112 of the assembly 1101 ca include an adhesive layer that can aid in selectively fixing the assembly 1101 around a target site or the bottom 1112 can receive or be integrally formed with a foam pad that includes an adhesive for selectively fixing the assembly 1101 around a target site.

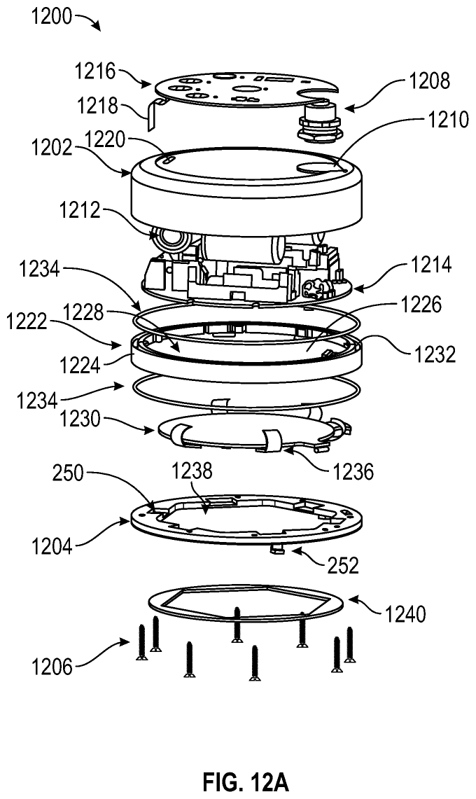

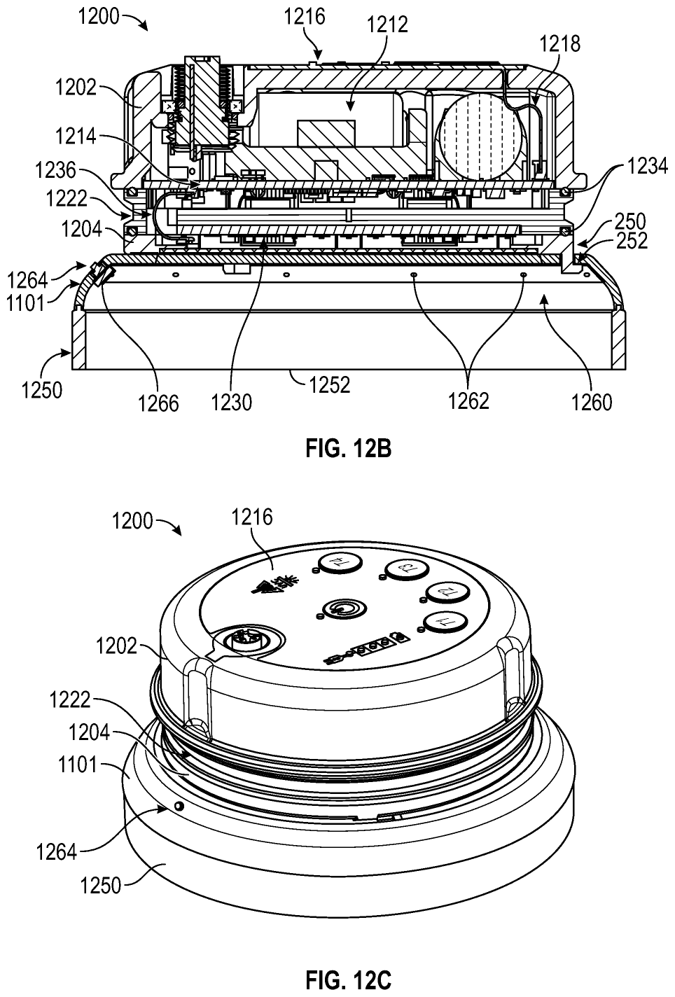

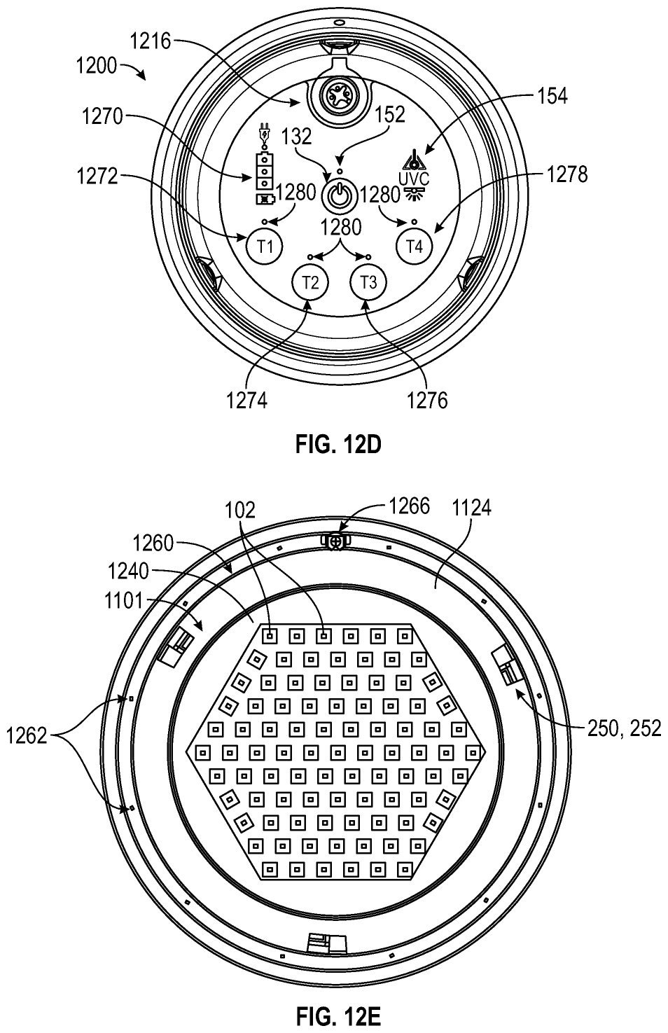

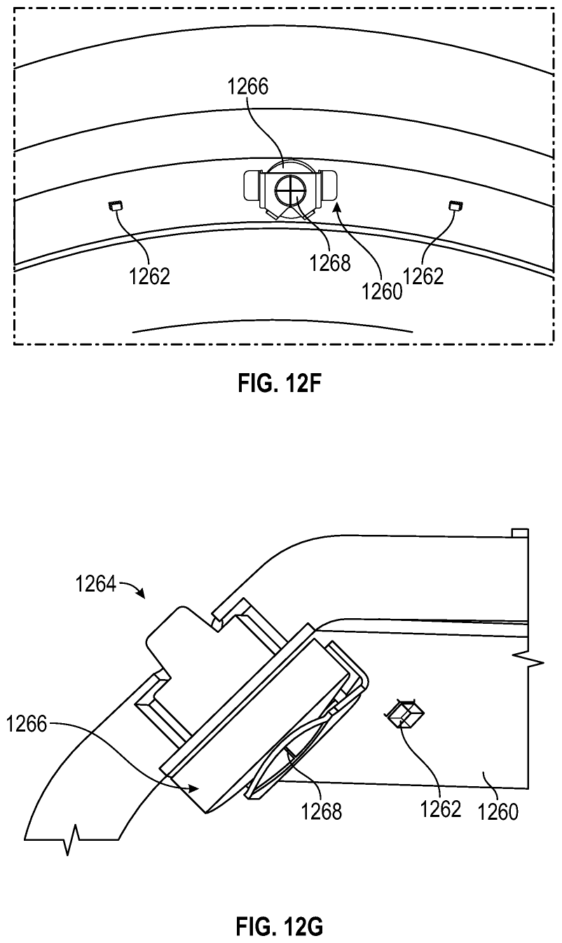

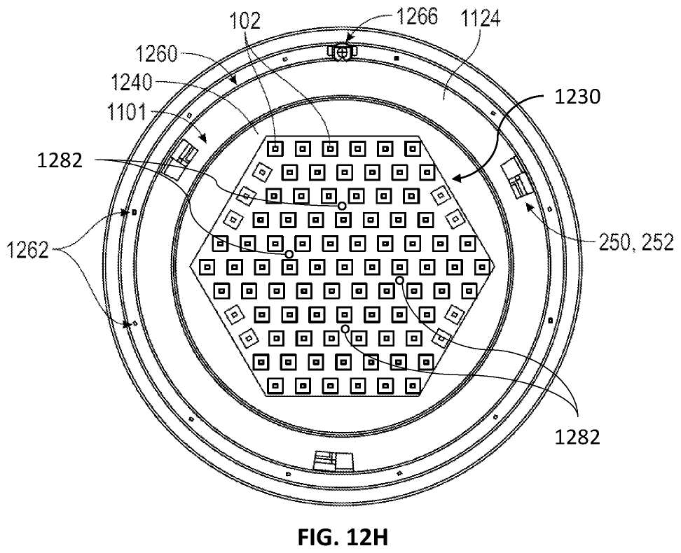

Embodiments of the assembly can also include LEDs, sensors and other components similar to those described herein with respect to the assemblies 601, 801, and/or with reference to FIGS. 12B, 12C, and 12E-H. Further, the assembly 1101 can have other configurations or shapes and/or can be different sizes.

With references to FIGS. 12A-H, there is illustrated another example embodiment of an optical head unit 210, which can be an exemplary assembly 1200 in accordance with embodiments of the present disclosure. The optical head unit 1200 can include the components of the optical head unit 210 described with reference to FIGS. 2A-B. A housing of the optical head unit 1200 can include a housing top 1202 and a housing bottom 1204 that can be secured together with fasteners 1206. A connector 1208 can extend through an opening 1210 in the housing top 1202 to provide power to batteries 1212 (e.g., power supply 106) and/or to communicate with a control board 1214 including at least a portion of the circuitry 100 described with reference to FIG. 1. For example, the control board 1214 can include the LED drivers 104 power supply 106, power and conditioning circuit 108, processor 110, memory 120, one or more of the sensors 140, and/or I/O devices 170 described with reference to FIG. 1. When the connector is not in use (e.g., during operation of the optical head unit), the connector port can be sealed. While the assembly 1200 is shown with connector 1208 and opening 1210 that may be sealed during operation, exemplary embodiments may be devoid of the connector 1208 and opening 1210 such that the assembly is self-contained, sealed, and does not include any ports or uncovered or unsealed openings. In such embodiments, the assembly 1200 can include components for wireless charging, such as an induction coil which can be disposed between a top surface of the housing top 1202 and the membrane 1216 or with the housing top 1202. By sealing the housing 1200 of the optical head unit such that there no openings, the sealed housing can prevent contamination of the interior of the housing from fluids (e.g., including bodily fluids); thereby advantageously allowing the exterior of the housing to be cleaned decontaminated between uses. Sealing the housing 1200 of the optical head unit also creates a thermodynamics challenge in preventing overheating of the circuitry within the interior of the housing 1200 (e.g., maintaining an operating temperature of the circuitry in the housing 1200 below 40° C.).

An interface membrane 1216 can be disposed on a top surface of the housing top 1202 and can include a ribbon cable 1218 that extends through a slit 1220 in the housing top 1202. The membrane 1210 can provide a user interface that allows a user to control and monitor an operation of the optical head unit 1200 and can include at least a portion of the circuitry 100 described with reference to FIG. 1. For example, the membrane can include power switch 132, indicators 150, and/or I/O devices 170. In the example embodiment shown in FIGS. 12A-D, the membrane can include an embodiments of the power switch 132, an embodiment of the power light 152, an embodiment of the in-operation light 154, and an embodiment of an indicator 1270 that indicates a charge remaining in the batteries 1212. The membrane can also include switches 1272-1278 for selecting preset operation timers and indicators in the form of lights 1280 for indicating which of the preset operation timers has been selected for use. As an example, the switch 1272 can be selected to set the during or operation of the optical head unit to a first preset timer, the switch 1274 can be selected to can be selected to set the during or operation of the optical head unit to a second preset timer, the switch 1276 can be selected to set the during or operation of the optical head unit to a third preset timer, and the switch 1278 can be selected to set the during or operation of the optical head unit to a fourth preset timer. While the membrane has been illustrated with switches to select preset timers, embodiments of the optical head unit can include different interfaces for controlling a duration of the operation of the optical head unit that allow the user to select preset timers and/or specify custom timers and/or embodiments of the optical head unit can automatically specify a timer based on a measured or know distance between the LEDs 102 and the target site. For embodiments in which the optical head unit automatically specifies the timer, the membrane 1216 can include an indicator, such as the display 158 to indicate the duration of operation based on the specified timer.

A heat sink 1222 can have a cylindrical outer ring 1224 and a planar circular surface or disc 1226 disposed at a bottom of the cylindrical outer ring 1224 to create an interior volume 1228 within which the control board 1214 can be received. The heat sink 1222 may be made of copper, aluminum, a combination thereof, or another thermal conductive material. The control board 1214 can be in physical contact with the heat sink 1222. For example, the control board 1214 can be circular in shape and rest on the planar circular surface 1226 of the heat sink 1222. A LED circuit board 1230 that includes the LEDs 102 (FIG. 1) can be disposed on the other side planar circular surface 1226 and can be in physical contact with the planar circular surface 1226. In some embodiments, a thermally conductive adhesive or gel can be disposed between the control board 1214 and the planar circular surface 1226 and/or between the LED circuit board 1230 and the planar circular surface 1226 to improve transfer of thermal energy from the control board 1214 and/or the LED circuit board 1230 to the heat sink 1222. In an exemplary non-limiting embodiment the heat sink 1222 may be made of copper and may utilize specialized thermal conductive technology, e.g., Super Pillar heat sink technology. Further the size, e.g., width or height, of the heat sink 1222 may be increased to allow for better thermal management. In an exemplary embodiment the heat sink 1222 may be the board of the LED circuit board 1230 whereby the LEDs and circuitry may be printed/mounted on the heat sink 1222, e.g., a Copper or Aluminum Super Pillar Base.

A top and bottom of the cylindrical outer ring 1224 can include a grooves 1232 for receiving gaskets; e.g., O-rings 1234, which can seal the housing of the optical head unit 1200 when it is assembled. In an assembled state, the O-rings 1234 can provide a fluid tight seal that prevent fluids from entering an interior of the housing. For example, the O-ring 1234 at the top of the cylindrical outer ring 1224 can be disposed between an interior surface of the housing top 1202 and the cylindrical outer ring 1224 can prevent fluid from entering an interior volume of the housing that includes the control board 1214 and batteries 1212, and the O-ring 1234 at the bottom of the cylindrical outer ring 1224 can be disposed between the bottom of the cylindrical outer ring 1224 and the interior surface of the housing bottom 1240 can prevent fluid from entering an interior volume of the housing that includes the LED circuit board 1230. Ribbon cables 1236 can provide electrical connections between the control board 1214 and the LED circuit board 1230. The ribbon cables 1236 can extend through openings in the planar circular surface 1226 of the heat sink 1222 when the housing of the optical head unit is assembled.

In an exemplary non-limiting embodiment, illustrated in FIG. 12H, the LED circuit board 1230 may be secured to the heat sink 1222 by one or more bolts 1282. The bolts 1282 may maximize contact and remove any gaps between the LED circuit 1230 and the heat sink 1222 to improve heat transfer between them and may be used in conjunction with or as an alternative to a thermally conductive adhesive or gel. The number and the position of the of bolts 1282 may be determined based on heat map modeling of the LED circuit board 1230 to optimize heat exchange. For example, the number, density, and output intensity of the LEDs 102 may necessitate In an embodiment there may be two pairs of bolts 1282 positioned around the center of the LED circuit board 1230 with each pair of bolts 1282 being equidistant the center of the LED circuit board 1230.

The housing bottom 1204 can include a combination of mating member 250 and 252 for mating with embodiments of the base depicted as assembly 1101 in the illustrated embodiment. An opening 1238 in the housing bottom 1204 allows light emitted from the LEDs on the LED circuit board 1230 to radiate out of the bottom of the optical head unit 1200. In the illustrated embodiment, the opening 1238 has a hexagonal shape, although the opening 1238 can have other shapes, such as rectangular, triangular, circular, triangular, and the like. A transparent lens 1240 can be fixed to a bottom surface of the housing bottom 1204 to seal the bottom of the optical head unit 1200 when in its assembled state. The lens 1240 can be a quartz/UV fused silica lens.

The assembly 1101 can include an LED circuit 1260 including LEDs 1262 disposed on an interior surface of the assembly. As an example, the LED circuit 1260 can be positioned on an interior side of assembly proximate to the top 1114, e.g., on or embedded in the interior side of the mounting surface 1224 or proximate to interior side of the mounting surface 1224. The portion of the interior surface upon which the LED circuit 1260 is disposed can be recessed to provide a track within which the LED circuit 1260 can be received. The LEDs 1262 can be positioned on the interior side of the assembly to emit light downwardly out of the bottom 1112 of the assembly to illuminate a target area. In a non-limiting example, the LEDs 1262 can emit light at one or more wavelengths in a range of approximately 400 nm to approximately 780 nm. The LED circuit 1260 can include a power switch 1264 to energize and de-energize the LEDs 1262 by selectively connecting and disconnecting a battery 1266 to the LEDs 1262. The power switch 1264 can include a light to indicate whether the LEDs 1262 are energized or de-energized. The power switch can be aligned with the battery 1266 and battery carrier 1268 to provide additional rigidity and strength under the power switch 1264 to mitigate a undesirable movement of the power switch 1264 when it is toggle or depressed to energize and/or de-energize the LEDs 1262.

The bottom 1112 of the assembly 1101 can be selective attached to or integrally formed with a foam ring 1250 having an adhesive layer 1252. The adhesive layer 1252 can covered with a protective layer that can be removed before positioning the assembly 1101 with the foam ring around a target site.

As shown in FIGS. 12B-C, when the housing is assembled the housing top 1202 and housing bottom 1204 can sandwich the cylindrical outer ring 1224 of the heat sink 1222 such that the cylindrical outer ring 1224 of the heat sink 1222 forms a portion of the exterior of the optical head unit 1200, while maintaining a fluid tight seal via the O-rings 1234 to prevent fluids from entering the interior of the optical head unit 1200 at the joints between the housing top and bottom and the heat sink 1222.

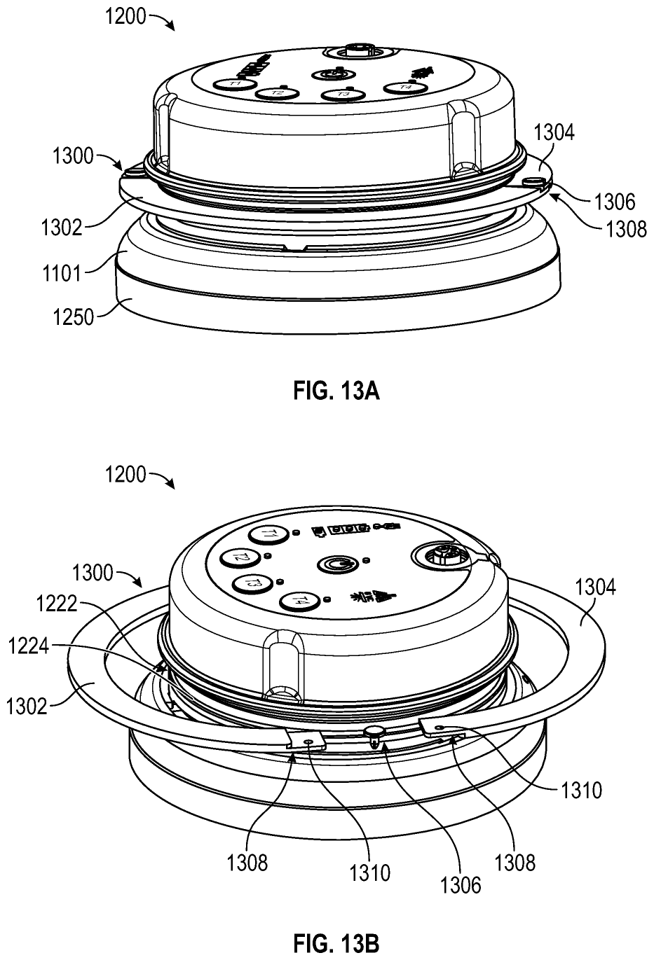

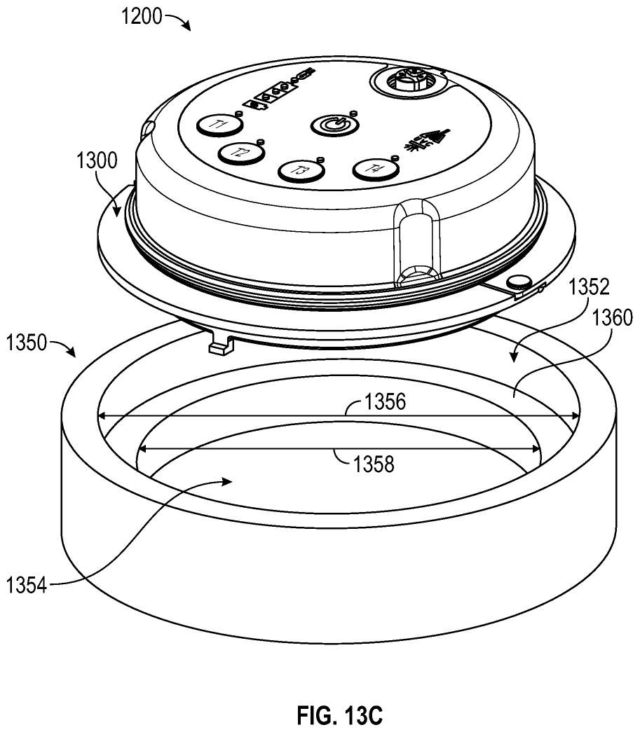

With reference to FIGS. 13A-B, an auxiliary thermally conductive ring 1300 can be coupled to an embodiment of the assembly 1200. For example, as shown in FIGS. 13A-B, the auxiliary thermally conductive ring can be removable coupled to the assembly 1200 to surround and be in physical contact with the outer cylindrical ring 1224 of the heat sink 1222 of the assembly 1200. The thermally conductive ring 1300 can be formed by segments 1302 and 1304 that can be secured to each other via a removable fastener 1306, such as a rivet. Lap joints 1308 can formed at the terminal ends of the segments 1302 and 1304 and can include openings 1310 through which the removable fastener 1306 can extend to secure the segments 1302 and 1304 together. The auxiliary thermally conductive ring can aid in the dissipation of heat from the assembly by provide additional surface area. The thermally conduct ring can be formed, for example, from aluminum, copper, a combination of aluminum and copper, and/or for other thermally conductive materials. The removable fastener can be formed, for example, from metal, plastic, rubber, and/or a combination thereof. Furthermore, while the example embodiments provides for a removable auxiliary thermally conduct ring, embodiments of the present disclosure can include an embodiments of the auxiliary thermal ring 1300 that is fixed to assembly 1200 and/or integrally formed with the heat sink 1222 of the assembly 1200.

FIG. 13C illustrates the assembly 1200 with the auxiliary thermally conductive ring 1300 and an example embodiment of a docking station 1350 configured to receive the assembly 1300 with the auxiliary thermally conductive ring 1300 in accordance with embodiments of the present disclosure. The docking station 1350 can be a storage caddy within which the assembly can be stored between uses. The docking station 1350 can also provide for charging and/or re-charging of the power supply of the optical head unit and/or for additional heat dissipation. For example, the docking station 1350 can provide for wireless power transfer as described herein (e.g., inductive charging, capacitive charging, RF charging, and/or any other forms of wireless power transfer) and/or can include a thermally conduct member that can be physical contact with the auxiliary ring 1300 and/or the heat sink 1222 when the assembly is stored in the docking station 1350.

The docking station 1350 can have a cylindrical housing with a stepped opening 1352 to an interior volume 1354 configured and dimensioned to receive the assembly 1200 with the auxiliary thermally conductive ring 1300. The stepped opening 1352 can have a first diameter 1356 and a second diameter 1358. A first portion of the stepped opening 1352 having the first diameter 1356 can extend from the top of the docking station 1350 down a first distance and can be configured and dimensioned to receive the assembly 1200 with the auxiliary thermally conductive ring 1300. A second portion of the stepped opening having the second diameter 1358 can be below the portion of the stepped opening 1352 having the first diameter 1356. That is, the second portion of the stepped opening corresponding to the second diameter can be recessed within the docking station 1350 such that the second portion begins from the first distance down from the top of the docking station 1350. The second portion of the stepped opening 1350 can be configured and dimensioned to receive a portion of the assembly 1200 below the auxiliary thermally conductive ring 1300, but the second diameter can be specified such that the auxiliary ring 1300 cannot pass through the second portion of the stepped opening 1352. Instead, when the assembly with the auxiliary ring 1300 is disposed in the docking station 1350, the auxiliary ring 1300 can abut a lateral, circumferential surface 1360 formed at the stepped transition from the first portion to the second portion of the stepped opening. The surface 1360 can have a width measured radially from the center axis of the opening that is equal to or greater than the width of the auxiliary ring 1300. The surface 1360 and/or the interior surface of the interior volume 1354 of the docking station 1350 can be formed from a thermally conduct material to aid in the dissipation of heat from the heat sink 1222.

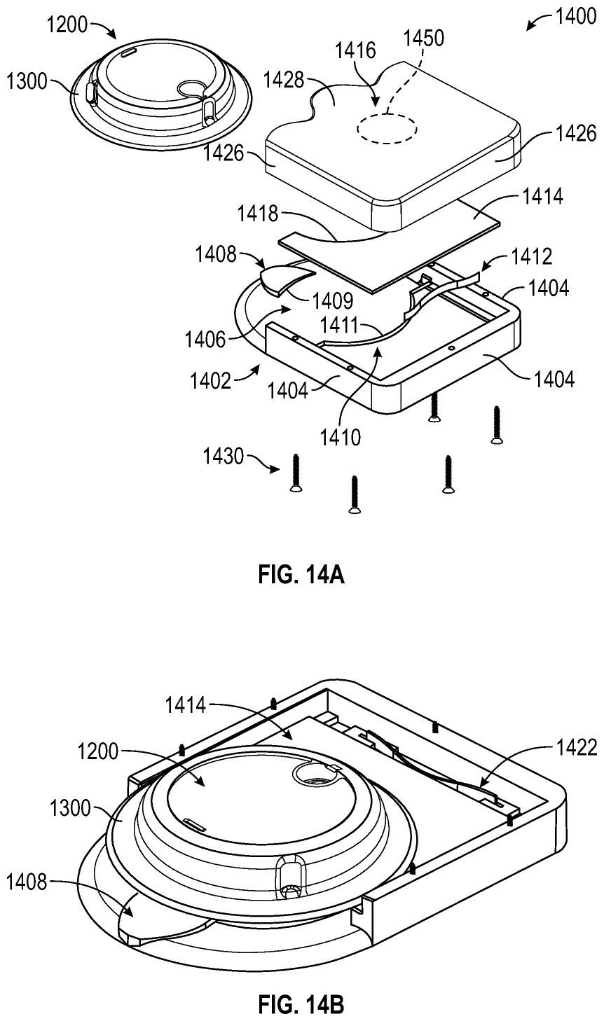

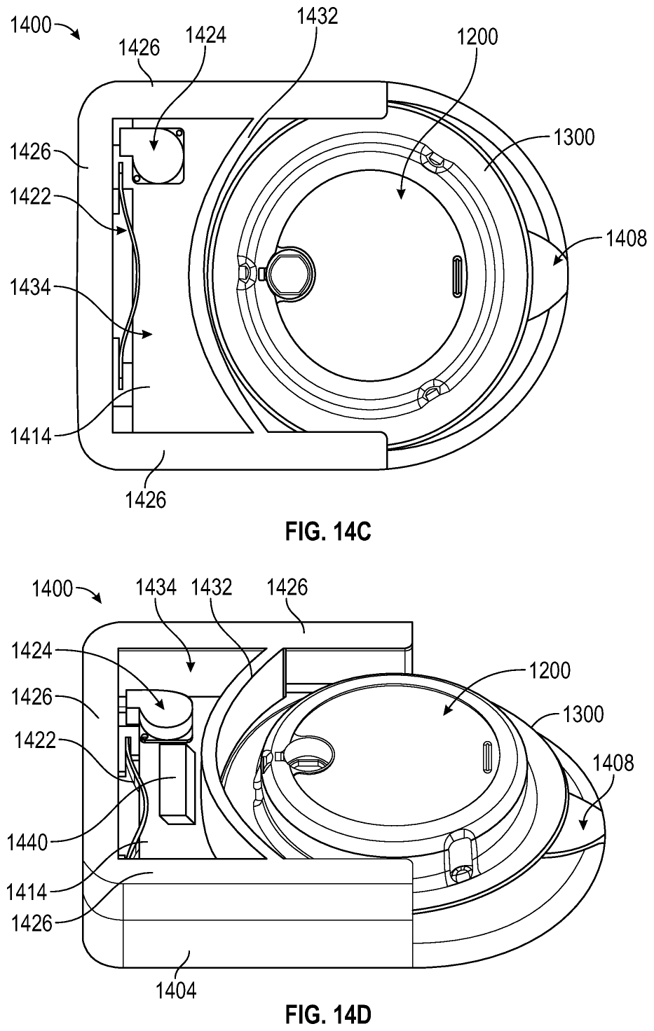

With reference to FIGS. 14A-D, there is illustrated example embodiment of a docking station 1400 for embodiments of the phototherapy systems. The docking station 1400 can be used to store the assembly 1200 between uses. The docking station 1400 can operate to charge/re-charge the batteries of embodiments of the optical head unit 210 (e.g., shown as assembly 1200) and/or to aid in cooling the optical head unit. As shown in a partially exploded view of FIG. 14A, a bottom member 1402 has side walls 1404 and includes a docking area 1406 configured and dimensioned to receive and retain the assembly 1200. The docking area 1406 can be defined in the bottom member 302 by a back retaining member 1408 defining a lip 1409 having a curvature that corresponds to the curvature of a bottom of the assembly 1200 and a front retaining member 1410 formed by a raised portion of the bottom member 1402 that defines lip 1411 that opposes the lip 1409 and has a curvature that corresponds to the curvature of a bottom of the assembly 1200. A resilient spring member 1412 can be position in proximity to the lip 1411 and can extend into the docking area 1406 and out from one of the side walls 1404 of the bottom member 1402. The resilient spring member 1412 can apply a force to the assembly 1200 when it is disposed in the docking area to urge the assembly toward the lip 1409 to aid in retaining the assembly 1200 in the docking area 1406. The resilient spring member 1412 can be actuated to remove the force being exerted onto the assembly to facilitate removal of the assembly 1200 from the docking area. A thermally conductive member 1414 can be positioned within an interior volume defined by the side walls 1404 and a top member 1416. The thermally conductive member 1414 can be a metal plate formed, for example, from aluminum, copper, a combination of aluminum and copper, and/or for other thermally conductive materials. A leading edge 1418 of the thermally conductive member can have a curvature that corresponds to the curvature of outer cylindrical ring 1224 of the heat sink 1222 on the assembly 1200. The remaining edges 1420 of the thermally conduct member 1414 can be linear. When the assembly 1200 is retained within the docking area 1406, the thermally conductive member 1414 can be in physical contact with at least a portion of the outer cylindrical ring 1224 of the heat sink 1422 to aid in dissipating heat from the interior of the optical head unit. For embodiments that include the thermally conductive auxiliary ring 1300, a bottom surface of the thermally conductive ring 1300 can abut and be in physical contact with the thermally conductive member 1414 of the docking station 140 to further aid in transferring heat away from the assembly 1200. A spring clip 1422 can be disposed between a back wall of the bottom member 1402 and the thermally conductive member 1414 to bias the thermally conduct member 1414 towards the front of the docking station 1400. When the optical head unit is retained in the docking area, the heat sink of the optical head unit can push against the thermally conductive member 1414 and a force exerted on the thermally conductive member 1414 by the spring clip 1422 can urge the thermally conductive member forward to maintain physical contact with the heat sink of the optical head unit. A fan 1424 can be disposed on the thermally conductive member 1414 and can be activated when the optical head unit is retained by the docking station 1400 and/or when a threshold temperature is exceed in the thermally conductive member 1414 or in the heat sink of the optical head unit.

The top member 1416 can include side walls 1426 and the top surface 1428. The side walls 1426 can generally align with and abut the top of the side walls 1404 of the bottom member 1402. The top member 1416 and bottom member 1402 can be secured together using fasteners 1430. The side walls 1426 and an inner wall 1432 of the top member 1416 can define an interior volume 1434 within which the electronics 1440 of the docking station 140 for controlling wireless power transfer and/the fan 1424 can be disposed and the top surface 1428 of the top member 1416 can include electrical components including an emitter 1450 (e.g., an inductive coil) to facilitate wireless charging of the power supply in the optical head unit (e.g., inductive charging, capacitive charging, RF charging, and/or any other forms of wireless power transfer). As an example, for embodiments that utilize inductive charging/re-charging of the power supply of the optical head unit, the top surface 1428 can include an induction coil that interacts with an induction coil of the power supply to charge/re-charge the power supply. As another example, for embodiments that utilize RF charging, the top surface 1428 can an RF charging emitter that interacts with an RF charging receiver of the power supply. Although the systems and methods of the present disclosure have been described with reference to exemplary embodiments thereof, the present disclosure is not limited to such exemplary embodiments and/or implementations. Rather, the systems and methods of the present disclosure are susceptible to many implementations and applications, as will be readily apparent to persons skilled in the art from the disclosure hereof. The present disclosure expressly encompasses such modifications, enhancements and/or variations of the disclosed embodiments. Since many changes could be made in the above construction and many widely different embodiments of this disclosure could be made without departing from the scope thereof, it is intended that all matter contained in the drawings and specification shall be interpreted as illustrative and not in a limiting sense. Additional modifications, changes, and substitutions are intended in the foregoing disclosure. Accordingly, it is appropriate that the appended claims be construed broadly and in a manner consistent with the scope of the disclosure.

What is claimed is:

1. A phototherapy system for treating and preventing infections in tissue of a human or animal, the system comprising:
an optical head unit having a housing incorporating circuitry, the circuitry including a plurality of light emitting diodes (LEDs) and a plurality of LED drivers, the LED drivers configured to control emission of light from the plurality of LEDs at one or more wavelengths in the light spectrum from approximately 100 nanometers to approximately 10,000 nanometers; and
a base that selectively and operatively couples to the optical head unit to provide a consistent distance to a target site and prevent the light emitted from the plurality of LEDs from escaping into an environment surrounding the target site.

2. The system of claim 1, wherein the plurality of LEDs cease to emit light in response to the optical head unit being detached from the base.

3. The system of claim 1, wherein the plurality of LEDs are controlled to emit the light for a specified period of time.

4. The system of claim 3, wherein the circuitry includes a distance sensor to determine a distance from the LEDs to the target site and a processor of the circuitry adjusts at least one of the specified time or an irradiance from the LEDs based on the determined distance to provide a specified dosage of energy to the target site.

5. The system of claim 1, wherein the plurality of LEDs are arranged in a lattice.

6. The system of claim 1, wherein the plurality of LEDs form a hexagonal perimeter.

7. The system of claim 1, wherein the base includes a shutter that controls an aperture through which the light emitted by the plurality of LEDs passes.

8. The system of claim 7, wherein the shutter controls at least one of a size of the aperture or a geometry of the aperture.

9. The system of claim 1, wherein the circuitry further comprises:
a non-transitory computer-readable medium storing instructions; and
a processor that executes the instructions to output drive signals to the LED drivers to control an operation of the plurality of LEDs.

10. The system of claim 1, wherein the circuitry further comprises:
one or more sensors to sense one or more parameters associated with an operation of the circuitry.

11. The system of claim 10, wherein the circuitry is configured to cease operation in response to the one or more sensors sensing that one of more of the parameters exceeds a specified threshold.

12. The system of claim 10, wherein the one or more sensors further comprise:
a temperature sensor to sense heat dissipated by the circuitry;
a light sensor to sense the light emitted by the plurality of LEDs; and
a contact sensor between the optical head unit and the base.

13. The system of claim 1, wherein the LED drivers provide variable current to one or more of the plurality of LEDs to irradiate the target site with a homogeneous distribution of the light emitted from the plurality of LEDs.

14. The system of claim 1, wherein the base has at least one of:
an asymmetrical C-shaped cross-sectional profile having an upper portion and a lower portion with the upper portion extending over the lower portion and extending further inwards than the lower portion to define the asymmetrical C-shaped cross-sectional profile; or
a generally truncated cone shape with a bottom opening having a first diameter and a top opening having a second diameter, the first diameter is greater than the second diameter.

15. The system of claim 14, wherein the base further comprises:
a light source to deliver light into the base;
wherein the base is configured to at least partially surround the target site and diffuse the light inwards and downwards toward the target site.

16. The system of claim 1, wherein different sets of the plurality of LEDs are controlled by different ones of the plurality of LED drivers.

17. The system of claim 1, wherein the plurality of LEDs are calibrated to emit the light with homogeneous light distribution with a spatial uniform density within ten percent (10%).

18. The system of claim 1, wherein at least one of a collimator or a filter is disposed over the plurality of LEDs.

19. The system of claim 1, wherein a temperature of a bottom of the base remains at less than 40° Celsius during operation of the circuitry.

20. The system of claim 1, wherein the one or more wavelengths at which one or more of the plurality of LEDs emit light is at least one of 235 nm or between approximately 207 nm and approximately 265 nm.

21. The system of claim 1, wherein the optical head unit further comprises:
a heat sink to transfer heat away from the circuitry.

22. The system of claim 21, wherein the housing of the optical head unit comprises:
a housing bottom;
a housing top; and
at least a portion of the heat sink disposed between the housing top and housing bottom, the at least a portion of the heat sink forming an exterior surface of the housing.

23. The system of claim 22, further comprising:
a thermally conductive auxiliary member configured to be removable attached to the at least a portion of the heat sink forming the exterior surface of the housing.

24. The system of claim 22, further comprising:
a docking station configured to receive the optical head unit, the docking station being configured to at least one of:
store the optical head unit between uses;
charge a power supply of the optical head unit; or
transfer heat away from the optical head unit.

25. The system of claim 24, wherein the optical head unit and the docking station are configured to charge the power supply using wireless power transfer.

26. The system of claim 24, wherein the docking station further comprises:
a docking area that receives and retains the optical head unit;
a thermally conduct member that is biased by a spring to be in physical contact with the at least a portion of the heat sink forming the exterior surface of the housing when the optical head unit is retained in the docking area.

27. The system of claim 26, wherein the docking station further comprises:
a fan disposed proximate to the thermally conductive member to cool the thermally conductive member.

28. The system of claim 21, further comprising:
at least one bolt securing the heat sink to the circuitry to improve the transfer of heat away from the circuitry.

29. The system of claim 28, wherein placement of the at least one bolt is determined based on heat map modeling of the circuitry.

30. The system of claim 1, wherein the housing of the optical head unit has a fluid tight seal.

31. The system of claim 1, further comprising a gel pad disposed on a bottom of the base to conform to contours of the tissue surrounding the target site.

32. The system of claim 1, wherein the light emitted by the plurality of LEDs irradiates the target site to achieve over ninety percent pathogen inactivation in under five minutes.

33. The system of claim 1, wherein the circuitry is removable and is removed from the housing and incorporated into a second housing of a second optical head unit;
wherein the optical head unit is decoupled from the base and the second optical head unit is selectively and operatively coupled to the base.

34. A phototherapy method for treating tissue of a human or animal at a target site, the method comprising:
connecting an optical head unit to a base;
disposing the base to surround a target site; and
controlling an output of a plurality of LEDs in the optical head unit to emit light through the base at one or more wavelengths in the light spectrum from approximately 100 nm to approximately 10,000 nm, the base preventing the light emitted from the plurality of LEDs from escaping into an environment surrounding a target site.

35. A phototherapy system for treating and preventing infections in tissue of a human or animal, the system comprising:
a light emitting diode (LED) that emits light at a wavelength of 235 nm to irradiate the tissue of a human or animal; and
an LED driver operatively coupled to the LED, the LED driver operable to cause the LED to emit the light at the wavelength of 235 nm for a specified period of time to deliver a specified dosage of the light to the tissue to decontaminate the tissue.

36. The phototherapy system of claim 32, further comprising:
a memory storing executable instructions; and
a processor operatively coupled to the memory and the LED driver, the processor is programmed to execute the instructions to:
select the specified period of time; and
control an operation of the LED driver to cause the LED to emit the light at the 235 nm wavelength for the specified period of time to deliver the specified dosage of the light.
